(19) United States
(12) Patent Application Publication
(10) Pub. No.: US 2024/0139349 A1
(43) Pub. Date: May 2, 2024

(54) GADOLINIUM CHELATE, PREPARATION METHOD THEREFOR, AND APPLICATION THEREOF

(71) Applicants: SUZHOU ZHECI PHARMACEUTICAL TECHNOLOGY CO., LTD., Suzhou, Jiangsu (CN); SOUTH MEDICAL UNIVERSITY, Guangzhou, Guangdong (CN)

(72) Inventors: Zheyu SHEN, Suzhou, Jiangsu (CN); Yudie LU, Suzhou, Jiangsu (CN)

(73) Assignees: SUZHOU ZHECI PHARMACEUTICAL TECHNOLOGY CO., LTD., Suzhou, Jiangsu (CN); SOUTH MEDICAL UNIVERSITY, Guangzhou, Guangdong (CN)

(21) Appl. No.: 18/279,528

(22) PCT Filed: Nov. 1, 2021
(86) PCT No.: PCT/CN2021/127915

§ 371 Date: Aug. 30, 2023

(30) Foreign Application Priority Data
Mar. 22, 2021 (CN) 202110303742.1

(51) Int. Cl. A61K 49/12

(52) U.S. Cl. CPC A61K 49/12

(57) ABSTRACT

A gadolinium chelate, a preparation method therefor, and an application thereof. The gadolinium chelate is a complex formed by chelation of gadolinium ions and macromolecules. The macromolecules comprise any one of or a copolymer or mixture of two or more of carboxylic acid-containing high-molecular polymers, amino-containing high-molecular polymers, hydroxyl-containing high-molecular polymers, polyester high-molecular polymers, polyether high-molecular polymers, polyamide high-molecular polymers, proteins, polypeptides, and polysaccharides. The gadolinium ions and the macromolecules form the gadolinium chelate by means of chelation. The gadolinium chelate is a water-soluble macromolecular drug, can improve a longitudinal relaxation rate r₁ and reduce a ratio of r₂/r₁, has good water-solubility and high stability, and meanwhile, can exhibit a good imaging effect in a short period of time and shorten an MRI time.

### CROSS-REFERENCE TO RELATED APPLICATIONS

The present application is a national phase entry under 35 USC § 371 of International Application PCT/CN2021/127915, filed Jan. 11, 2021, which claims the benefit of and priority to Chinese Patent Application No. 202110303742.1, filed Mar. 22, 2021, the entire disclosures of which are incorporated herein by reference.

### TECHNICAL FIELD

The present disclosure relates to the technical field of biomedical materials, and in particular to a gadolinium chelate, a preparation method and application thereof.

### BACKGROUND

Magnetic resonance imaging (MRI) can be used for detecting small or specific lesions sensitively, thus reducing the rates of missed diagnosis or erroneous diagnosis. As a chemical agent capable of improving the quality of MRI images, an MRI contrast agent has become an important supplement to MRI examinations. T₁ (longitudinal relaxation time) and T₂ (transverse relaxation time) are important tissue feature parameters of MRI. When paramagnetic substances (such as gadolinium, iron and manganese) in a sample approach hydrogen atoms in resonance, the magnetic field where protons are located can be effectively changed, the tissue relaxation times T₁ and T₂ can be shortened, signals in an image enhancement region are thus enhanced, and the signal-to-noise ratio (ΔSNR) of the tissue can be improved. Using contrast agents to improve the contrast ratio of MRI tissue exactly takes advantage of this function. The range of clinical application of MRI techniques is also greatly widened thanks to the addition of MRI contrast agents, and the MRI techniques have a very important application value in the field of clinical diagnosis.

Clinically, T₁-weighted imaging, T₂-weighted imaging, and T₂*-weighted imaging are the most commonly used imaging sequences to obtain tissue images. Correspondingly, MRI contrast agents are mainly classified into two types according to their enhancement characteristics: a positive contrast agent (T₁ contrast agent) and a negative contrast agent (T₂ contrast agent). Since the T₂ contrast agent has the features such as darker imaging and likely generated magnetic susceptibility artifacts, while the T₁ contrast agent has a more prominent effect on brightening lesion areas in images, the T₁ contrast agent exhibits a very obvious advantage in soft tissue detection. A relaxation rate including longitudinal relaxation rate r₁, and transverse relaxation rate r₂, is one of the key evaluation indexes to measure the performance of the MRI contrast agents. Generally, if an r₁ value is larger and an r₂/r₁ value is smaller, it is more conducive to T₁-weighted imaging, and the signal-to-noise ratio of images is higher under the condition of the same dose; conversely, if the r₂ value is larger and the r₂/r₁ value is larger, it is more conducive to T₂-weighted imaging. At present, commercially available T₁ contrast agents are mainly gadolinium-based contrast agents, including cyclic chelates and linear chelates. The cyclic chelates include, for example, Gadavist® (Gd-BT-DO3A), Dotarem® (Gd-DOTA) and ProHance® (Gd-HP-DO3A), etc., and the linear chelates include, for example, Magnevist® (Gd-DTPA), Omniscan® (Gd-DTPA-BMA), Primovist® (Gd-EOB-DTPA) and Multihance® (Gd-BOTPA), etc. These commercially available gadolinium-based contrast agents are micromolecular, which exhibit very limited relaxation properties, and provide r₁ values in the range of 4-7 mM⁻¹ s⁻¹.

Since both linear and macrocyclic gadolinium-based contrast agents cause trace gadolinium deposition in the brain and other tissues, if high doses of gadolinium-based contrast agents are used in clinical scanning to enhance the contrast between normal tissues and diseased tissues, it may result in potential risks such as nephrotoxicity in some patient populations. Currently, in order to take into account higher-sensitivity lesion detection and dose limitation, the contrast agents for clinical diagnosis are usually dosed at a gram level. Decreasing the dose of a contrast agent used is a very desirable way to improve the signal-to-noise ratio of an image, because a compound having a high relaxation can be detected at a low dose, or present a greater contrast to a compound having a low relaxation at an equivalent dose, making a contrast effect better.

### SUMMARY

The present disclosure aims to solve at least one of the technical problems existing in the prior art. In view of this, the present disclosure provides a gadolinium chelate, which has an extremely high longitudinal relaxation rate r₁ and an extremely low r₂/r₁ ratio, good water solubility and high stability, and can exhibit a good imaging effect in a short time and shorten an MRI time.

The present disclosure further provides a preparation method and an application of the gadolinium chelate.

Specifically, the technical solution used in the present disclosure is as follows.

In a first aspect, the present disclosure provides a gadolinium chelate. The gadolinium chelate is a complex formed by chelation of gadolinium ions and a macromolecule, wherein the macromolecule includes any one of or a copolymer or mixture of two or more of a carboxylic acid-containing high-molecular polymer, an amino-containing high-molecular polymer, a hydroxyl-containing high-molecular polymer, a polyester high-molecular polymer, a polyether high-molecular polymer, a polyamide high-molecular polymer, a protein, a polypeptide, or a polysaccharide.

According to the first aspect, the gadolinium chelate of the present disclosure has at least the following beneficial effects.

In the present disclosure, the gadolinium ions and the macromolecule chelate to form the gadolinium chelate that is a water-soluble macromolecular drug. Compared with the network crosslinked structures or core-shell structures formed by wrapping gadolinium ions or gadolinium oxides with macromolecules in the related art, the longitudinal relaxation rate r₁ of the gadolinium chelate can be increased, the r₂/r₁ ratio thereof can be decreased, and the gadolinium chelate have a good water solubility and high stability, and exhibit a good imaging effect in a short time and shorten an MRI time.

In some embodiments of the present disclosure, a molecular weight of the macromolecule is 1,000-1,000,000, and suitable macromolecules may be selected according to a specific circumstance.

In some embodiments of the present disclosure, the carboxylic acid-containing high-molecular polymer includes any one or more selected from the group consisting of polyacrylic acid, polymaleic acid, polymethacrylic acid, poly(2-ethylacrylic acid), polyglutamic acid, and polyaspartic acid.

In some embodiments of the present disclosure, the amino-containing high-molecular polymer includes any one or more selected from the group consisting of polylysine, polyarginine, polyhistidine, and polyethyleneimine.

In some embodiments of the present disclosure, the hydroxyl-containing high-molecular polymer includes any one or more selected from the group consisting of polyserine, polythreonine, polytyrosine, and polyvinyl alcohol.

In some embodiments of the present disclosure, the polyester high-molecular polymer includes any one or more selected from the group consisting of polylactic acid, polyglycolic acid, polycaprolactone, and poly(2-hydroxyethyl methacrylate).

In some embodiments of the present disclosure, the polyether high-molecular polymer includes any one or more selected from the group consisting of polyethylene glycol, polypropylene glycol, and polytetramethylene glycol.

In some embodiments of the present disclosure, the polyamide high-molecular polymer includes any one or more selected from the group consisting of polyglutamine, polyasparagine, polyacrylamide, and polymethacrylamide.

In some embodiments of the present disclosure, the protein includes any one or more selected from the group consisting of a human protein, an animal protein, a plant protein, and a recombinant protein.

In some embodiments of the present disclosure, the polypeptide includes any one or more selected from the group consisting of an RGD peptide and a β-amyloid polypeptide.

In some embodiments of the present disclosure, the polysaccharide includes any one or more selected from the group consisting of chitosan and sodium alginate.

In some embodiments of the present disclosure, the gadolinium ions are trivalent gadolinium ions.

In a second aspect, the present disclosure provides a preparation method of the above-mentioned gadolinium chelate, comprising: subjecting gadolinium ions and a macromolecule to a chelation reaction to obtain the gadolinium chelate.

More specifically, the preparation method of the above-mentioned gadolinium chelate, comprising: mixing a gadolinium ion solution with a macromolecular solution to perform a chelation reaction, so as to obtain a gadolinium chelate.

In some embodiments of the present disclosure, a concentration of the gadolinium ion solution is 10-1000 mM, preferably 50-250 mM.

In some embodiments of the present disclosure, a concentration of the macromolecular solution is 0.1-50 mg/mL, preferably 2.0-10 mg/mL.

In some embodiments of the present disclosure, a volume ratio of the macromolecular solution to the gadolinium ion solution is (1-1000):1, preferably (25-50):1.

In some embodiments of the present disclosure, both the gadolinium ion solution and the macromolecular solution are aqueous solutions.

In some embodiments of the present disclosure, the gadolinium ion solution includes any one or more selected from the group consisting of a gadolinium nitrate solution, a gadolinium fluoride solution, a gadolinium chloride solution, a gadolinium bromide solution and a gadolinium iodide solution, preferably the gadolinium nitrate or gadolinium chloride solution.

In some embodiments of the present disclosure, a pH of a reaction solution obtained by mixing the gadolinium ion solution and the macromolecular solution is 2.0-12.0, preferably about 10.

In some embodiments of the present disclosure, the reaction is conducted at a temperature of 25° C.-100° C., preferably 100° C.

In some embodiments of the present disclosure, the reaction is lasts for more than 10 min, preferably 30-120 min.

In a third aspect, the present disclosure provides an application of the above-mentioned gadolinium chelate in the preparation of a magnetic resonance imaging contrast agent (MRI contrast agent).

Compared with the prior art, the present disclosure has the following beneficial effects.

The gadolinium chelate of the present disclosure has an extremely high r₁ value (>80 mM⁻¹ s⁻¹, 1.5 T; >29 mM⁻¹ s⁻¹, 3.0 T), which is much higher than that (r₁=4-7 mM⁻¹ s⁻¹, 1.5 T) of the MRI contrast agent commonly used in clinical practice, and is also higher than that (>50 mM⁻¹ s⁻¹, 1.5 T) of the MRI contrast agent obtained by coating a gadolinium oxide with a high molecular polymer, and that (14 mM⁻¹ s⁻¹, 3.0 T) of a polymer network microsphere contrast agent prepared from gadolinium ions and a polymer used in the related art, and the gadolinium chelate has an extremely low r₂/r₁ ratio (r₂/r₁<2.0, 1.5 T). As a novel MRI contrast agent, the gadolinium chelate of the present disclosure can not only significantly improve the signal-to-noise ratio of MRI images, but also has the advantages of excellent water solubility, ultra-low release rate of gadolinium (III) ions within 7 days, being stable and effective, etc. Moreover, the gadolinium chelate of the present disclosure has a rapid imaging effect at a low dose, a shortened imaging time, and strong practicability.

### BRIEF DESCRIPTION OF DRAWINGS

FIG. 1A shows a longitudinal relaxation rate of Gd-PAA of Example 1; and FIG. 1B shows a transverse relaxation rate of Gd-PAA of Example 1;

FIG. 2 shows a gadolinium ion release rate of Gd-PAA of Example 1 under the condition of pH=7.4 for 1-7 days;

FIG. 3A shows T₁-weighted MRI images of Gd-PAA of Example 1, a commercially available contrast agent Gadavist and pure water, wherein TR=300 ms and TE=6.5 ms; FIG. 3B shows T₁-weighted MRI images of Gd-PAA of Example 1, a commercially available contrast agent Gadavist and pure water, wherein TR=100 ms and TE=6.5 ms; FIG. 3C shows the relative MRI signal intensities of Gd-PAA of Example 1, a commercially available contrast agent Gadavist and the pure water, ****p<0.0001;

FIG. 4 is an infrared absorption spectrogram of Gd-PAA prepared in Example 1;

FIG. 5A is a MRI image showing a tumor-bearing mice before injecting with Gd-PAA prepared in Example 1; FIG. 5B is a MRI image showing a tumor-bearing mice obtained at 15 minutes after injecting with Gd-PAA prepared in Example 1; FIG. 5C is a MRI image showing a tumor-bearing mice obtained at 30 minutes after injecting with Gd-PAA prepared in Example 1; and FIG. 5D is a MRI image showing a tumor-bearing mice obtained at 45 minutes after injecting with Gd-PAA prepared in Example 1;

FIG. 6A shows a longitudinal relaxation rate of Gd-PASP of Example 2; and FIG. 6B shows a transverse relaxation rate of Gd-PASP of Example 2;

FIG. 7 shows a gadolinium ion release rate of Gd-PASP of Example 2 under the condition of pH=7.4 for 1-7 days;

FIG. 8A shows T₁-weighted MRI images of Gd-PASP of Example 2, a commercially available contrast agent Gadavist and pure water, wherein TR=300 ms and TE=6.5 ms; FIG. 8B shows T₁-weighted MRI images of Gd-PASP of Example 2, a commercially available contrast agent Gadavist and pure water, wherein TR=100 ms and TE=6.5 ms; and FIG. 8C shows the relative MRI signal intensities of Gd-PASP of Example 2, a commercially available contrast agent Gadavist and the pure water, ****p<0.0001;

FIG. 9A shows a longitudinal relaxation rate of Gd-HPMA of Example 3; and FIG. 9B shows a transverse relaxation rate of Gd-HPMA of Example 3; FIG. 10A shows a longitudinal relaxation rate of Gd-PMAA of Example 4; and FIG. 10B shows a transverse relaxation rate of Gd-PMAA of Example 4;

FIG. 11A shows a longitudinal relaxation rate of Gd-PEAA of Example 5; and FIG. 11B shows a transverse relaxation rate of Gd-PEAA of Example 5;

FIG. 12A shows a longitudinal relaxation rate of Gd-γ-PGA of Example 6; and FIG. 12B shows a transverse relaxation rate of Gd-γ-PGA of Example 6;

FIG. 13A shows a longitudinal relaxation rate of Gd-ε-PL of Example 7; and FIG. 13B shows a transverse relaxation rate of Gd-ε-PL of Example 7;

FIG. 14A shows a longitudinal relaxation rate of Gd-PLR of Example 8; and FIG. 14B shows a transverse relaxation rate of Gd-PLR of Example 8;

FIG. 15A shows a longitudinal relaxation rate of Gd-PLH of Example 9; and FIG. 15B shows a transverse relaxation rate of Gd-PLH of Example 9;

FIG. 16A shows a longitudinal relaxation rate of Gd-PEI of Example 10; and FIG. 16B shows a transverse relaxation rate of Gd-PEI of Example 10;

FIG. 17A shows a longitudinal relaxation rate of Gd-PSer of Example 11; and FIG. 17B shows a transverse relaxation rate of Gd-PSer of Example 11 FIG. 18A shows a longitudinal relaxation rate of Gd-PThr of Example 12; and FIG. 18B shows a transverse relaxation rate of Gd-PThr of Example 12;

FIG. 19A shows a longitudinal relaxation rate of Gd-PTyr of Example 13; and FIG. 19B shows a transverse relaxation rate of Gd-PTyr of Example 13;

FIG. 20A shows a longitudinal relaxation rate of Gd-PVA of Example 14; and FIG. 20B shows a transverse relaxation rate of Gd-PVA of Example 14;

FIG. 21A shows a longitudinal relaxation rate of Gd-PLA of Example 15; and FIG. 21B shows a transverse relaxation rate of Gd-PLA of Example 15;

FIG. 22A shows a longitudinal relaxation rate of Gd-PGA of Example 16; and FIG. 22B shows a transverse relaxation rate of Gd-PGA of Example 16;

FIG. 23A shows a longitudinal relaxation rate of Gd-PCL of Example 17; and FIG. 23B shows a transverse relaxation rate of Gd-PCL of Example 17;

FIG. 24A shows a longitudinal relaxation rate of Gd-PHEMA of Example 18; and FIG. 24B shows a transverse relaxation rate of Gd-PHEMA of Example 18;

FIG. 25A shows a longitudinal relaxation rate of Gd-PEG of Example 19; and FIG. 25B shows a transverse relaxation rate of Gd-PEG of Example 19;

FIG. 26A shows a longitudinal relaxation rate of Gd-PPG of Example 20; and FIG. 26B shows a transverse relaxation rate of Gd-PPG of Example 20;

FIG. 27A shows a longitudinal relaxation rate of Gd-PTMG of Example 21; and FIG. 27B shows a transverse relaxation rate of Gd-PTMG of Example 21;

FIG. 28A shows a longitudinal relaxation rate of Gd-PolyQ of Example 22; and FIG. 28B shows a transverse relaxation rate of Gd-PolyQ of Example 22;

FIG. 29A shows a longitudinal relaxation rate of Gd-PHEA of Example 23; and FIG. 29B shows a transverse relaxation rate of Gd-PHEA of Example 23;

FIG. 30A shows a longitudinal relaxation rate of Gd-PAM of Example 24; and FIG. 30B shows a transverse relaxation rate of Gd-PAM of Example 24;

FIG. 31A shows a longitudinal relaxation rate of Gd-PMAM of Example 25; and FIG. 31B shows a transverse relaxation rate of Gd-PMAM of Example 25;

FIG. 32A shows a longitudinal relaxation rate of Gd-HSA of Example 26; and FIG. 32B shows a transverse relaxation rate of Gd-HSA of Example 26;

FIG. 33A shows a longitudinal relaxation rate of Gd-BSA of Example 27; and FIG. 33B shows a transverse relaxation rate of Gd-BSA of Example 27;

FIG. 34A shows a longitudinal relaxation rate of Gd-RGD of Example 28; and FIG. 34B shows a transverse relaxation rate of Gd-RGD of Example 28;

FIG. 35A shows a longitudinal relaxation rate of Gd-AP of Example 29; and FIG. 35B shows a transverse relaxation rate of Gd-AB of Example 29;

FIG. 36A shows a longitudinal relaxation rate of Gd-CS of Example 30; and FIG. 36B shows a transverse relaxation rate of Gd-CS of Example 30;

FIG. 37A shows a longitudinal relaxation rate of Gd-SA of Example 31; and FIG. 37B shows a transverse relaxation rate of Gd-SA of Example 31;

FIG. 38A shows a longitudinal relaxation rate of Gd-PAA-PLA of Example 32; and FIG. 38B shows a transverse relaxation rate of Gd-PAA-PLA of Example 32;

FIG. 39A shows a longitudinal relaxation rate of Gd-PGA-PEG of Example 33; and FIG. 39B shows a transverse relaxation rate of Gd-PGA-PEG of Example 33;

FIG. 40A shows a longitudinal relaxation rate of Gd-PAA/PASP of Example 34; and FIG. 40B shows a transverse relaxation rate of Gd-PAA/PASP of Example 34; and

FIG. 41A shows a longitudinal relaxation rate of Gd-γ-PGA/PASP of Example 35; and FIG. 41B shows a transverse relaxation rate of Gd-γ-PGA/PASP of Example 35.

### DETAILED DESCRIPTION

The technical solutions of the present disclosure will be further described in conjunction with the following specific examples.

### Example 1

Preparation of Gadolinium Chelate (Gd-PAA)

40 mL of polyacrylic acid (molecular weight (M_{w})=2000) solution with a concentration of 4.0 mg/mL was prepared, and the polymer solution was heated at 100° C. for refluxing. 0.8 mL of gadolinium nitrate solution with a concentration of 125 mM was immediately added to the reaction system, and the reaction continued for 60 minutes at 100° C. under the magnetic stirring. After being cooled to the room temperature, the solution was purified by means of membrane dialysis to obtain a product labeled as Gd-PAA finally.

Characterization and performance tests were performed on a sample provided in Example 1. The gadolinium recovery rate of the sample prepared in Example 1 was calculated, and it was higher than 90%, indicating that a chelating effect was good and the utilization rate of raw materials was high. The sample prepared in Example 1, and the commercially available contrast agents (Magnevist, and Gadavist), and a gadolinium nitrate solution were each prepared into at least 5 aqueous solutions of different concentrations, in vitro imaging was performed by means of 1.5 T and 3.0 T clinical MRI systems and a 7.0 T small animal MRI system, and longitudinal relaxation times and transverse relaxation times (T₁, T₂) were determined. A longitudinal relaxation rate and a transverse relaxation rate (r₁, r₂) were calculated by the following formula (c represents the concentration of a magnetic substance in the contrast agent, T represents the relaxation time, where i=1 or 2). The results are shown in Table 1.

$${r}_{i} = \frac{Δ ( 1 / {T}_{i} )}{Δ c}$$

TABLE 1
Synthesis of Gd-PAA and MRI characterization results

| | Gadolinium | | | | |
|---|---|---|---|---|---|
| | recovery rate | | | | |
| Sample | (%) | H₀ (T) | r₁ (mM⁻¹ s⁻¹) | r₂ (mM⁻¹ s⁻¹) | r₂/r₁ |
| Gd-PAA | 95.5 | 1.5 | 82.76 ± 0.81 | 92.46 ± 0.58 | 1.12 ± 0.00 |
| | | 3.0 | 56.23 ± 1.69 | 86.58 ± 1.18 | 1.54 ± 0.03 |
| | | 7.0 | 17.18 | 92.55 | 5.39 |
| Gadavist | — | 3.0 | 4.60 ± 0.09 | 4.90 ± 0.04 | 1.07 ± 0.03 |
| | | 7.0 | 3.77 | 5.56 | 1.47 |
| Magnevist | — | 3.0 | 4.88 ± 0.12 | 5.00 ± 0.10 | 1.03 ± 0.03 |
| | | 7.0 | 3.68 | 5.38 | 1.46 |
| Gd(NO₃)₃ | — | 3.0 | 11.39 ± 0.25 | 12.04 ± 0.31 | 1.06 ± 0.006 |
| | | 7.0 | 8.92 | 12.90 | 1.45 |

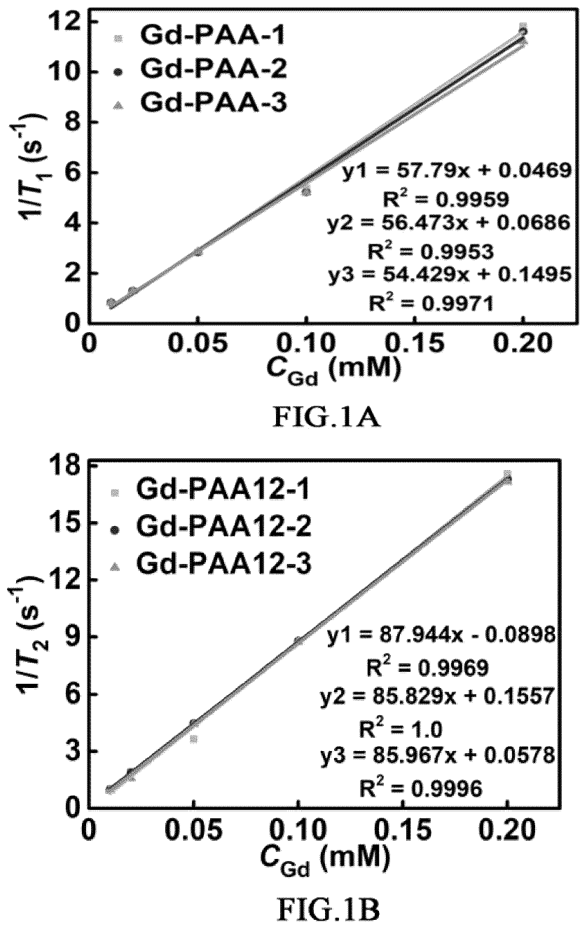

Specifically, FIG. 1A and FIG. 1B are charts showing relaxation rates of the samples Gd-PAA of Example 1 when used as an MRI contract agent (three samples Gd-PAA-1, Gd-PAA-2 and Gd-PAA-3 were subjected to a parallel test for three times), and in the relationship that 1/Tᵢ changes with the gadolinium concentration, a slope of the fitted straight line is the Tᵢ relaxation rate (i=1 or 2). It could be seen that in the 3.0 T clinical MRI scanning system (Philips, Ingenia), the longitudinal relaxation rate of the gadolinium chelate Gd-PAA is 56.23±1.69 mM⁻¹ s⁻¹, which is more than ten times the r₁ value (r₁=4-7 mM⁻¹ s⁻¹) of the commercially available gadolinium-based contrast agent, and significantly higher than that of a highly toxic free gadolinium ion solution. In addition, the Gd-PAA has r₁ value tested by the 1.5 T MRI scanning system of 82.76±0.81 mM⁻¹ s⁻¹, and also has an extremely low r₂/r₁ ratio (1.12±0.00), indicating that the MRI contrast effect is particularly excellent.

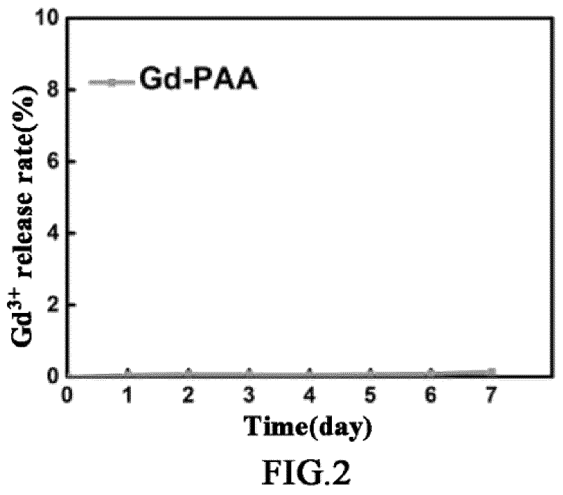

FIG. 2 shows a gadolinium (III) ion release rate of the sample Gd-PAA under the condition of pH=7.4 for 1-7 days. The sample Gd-PAA hardly releases ions (<0.2%), indicating that the sample provided in Example 1 has high stability when used as the MRI contrast agent.

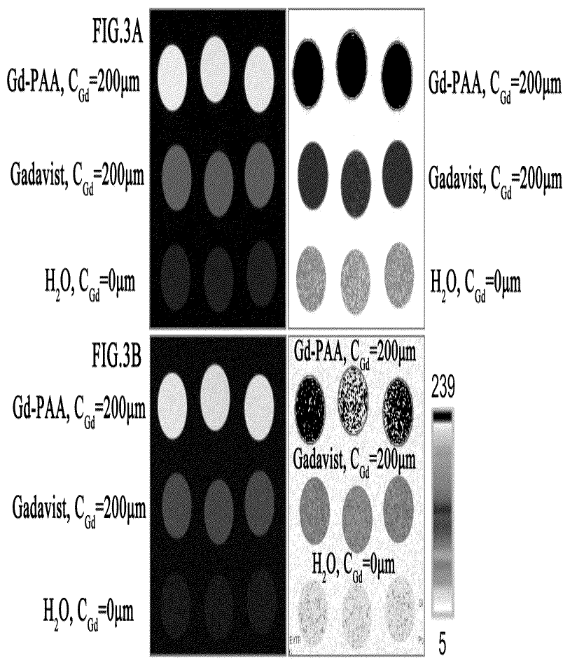

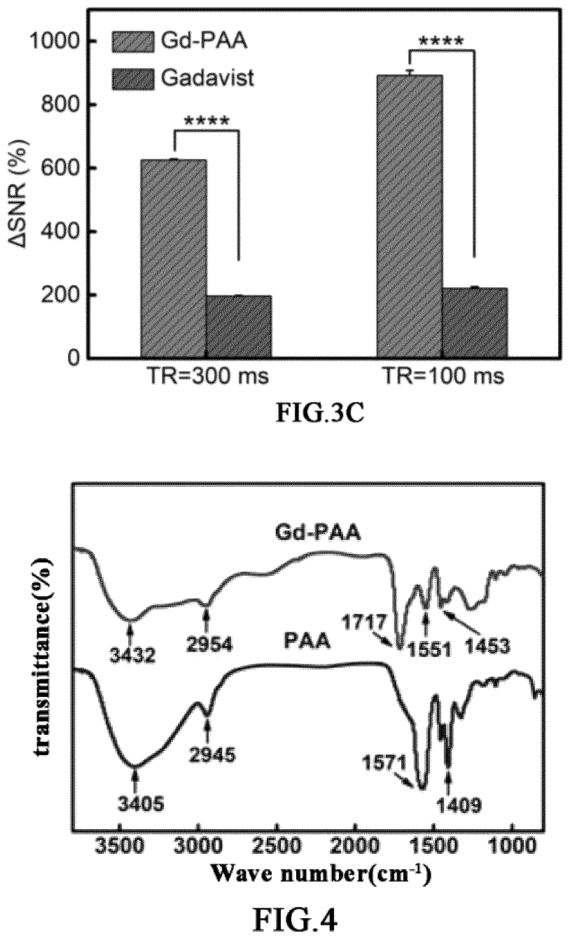

On the basis of the r₁ value, the r₂/r₁ ratio in Table 1 and the gadolinium ion release rate, taking the sample Gd-PAA as a typical representative, T1-weighted MRI images of Gd-PAA, the commercially available contrast agent Gadavist and the pure water under a 7.0 T magnetic field are shown in FIG. 3A and FIG. 3B, wherein TR=300 ms and TE=6.5 ms in FIG. 3A, TR=100 ms and TE=6.5 ms in FIG. 3B, and FIG. 3C shows the relative MRI signal intensities of Gd-PAA, Gadavist and the pure water, ****p<0.0001. The results show that the MRI signal intensity of Gd-PAA is much higher than that of the commercial contrast agent Gadavist.

Referring to FIG. 4, it is an infrared absorption spectrogram of the typical sample Gd-PAA. A strong and broad absorption peak of polyacrylic acid at 3405 cm⁻¹ is an O-H stretching vibration peak, while an O-H stretching vibration peak of the sample Gd-PAA moves to 3432 cm⁻¹, the absorption peak is narrowed, and its intensity is weakened, indicating that an O-H and a hydrogen bond at this position breaks and reacts with Gd³⁺. The polyacrylic acid has antisymmetric and symmetric stretching vibration absorption peaks of carboxyl groups at 1571 cm⁻¹ and 1409 cm⁻¹. The sample Gd-PAA has a new C═O stretching vibration absorption peak at 1717 cm⁻¹, indicating that oxygen and gadolinium ions are coordinate in the carboxyl group of the polyacrylic acid, thus proving the formation of the gadolinium chelate Gd-PAA.

FIG. 5A, FIG. 5B, FIG. 5C and FIG. 5D show MRI images of a tumor-bearing mice suffered from tail vein injection with the Gd-PAA chelate in different time periods (with an injection dose of 5 mg/kg). Wherein FIG. 5A is an MRI image obtained before tail vein injection, FIG. 5B is an MRI image obtained at 15 minutes after tail vein injection, FIG. 5C is an MRI image obtained at 30 minutes after tail vein injection, and FIG. 5D is an MRI image obtained at 45 minutes after injection of the Gd-PAA chelate. It can be seen that an MRI signal of the tumor site is the strongest at 30 minutes after intravenous injection, indicating that this macromolecular chelate can not only remarkably improve the imaging contrast ratio of tumor tissue, but also has a shorter imaging time, and is conducive to clinical promotion.

### Example 2

Preparation of Gadolinium Chelate (Gd-PASP)

40 mL of polyaspartic acid (M_{w}=2000) solution with a concentration of 4.0 mg/mL was prepared, and the polymer solution was heated at 100° C. for refluxing. 0.8 mL of gadolinium nitrate solution with a concentration of 125 mM was immediately added to the reaction system, and the reaction continued for 60 minutes at 100° C. under the magnetic stirring. After being cooled to the room temperature, the solution was purified by means of membrane dialysis to obtain a product labeled as Gd-PASP finally.

Characterization and performance tests were performed on a sample provided in Example 2. The gadolinium recovery rate of the sample prepared in Example 2 was higher than 90%, indicating that the utilization rate of raw materials was high. The sample prepared in Example 2 was prepared into at least 5 aqueous solutions of different concentrations, in vitro imaging was performed by means of the 3.0 T clinical MRI system and the 7.0 T MRI system, and a longitudinal relaxation time and a transverse relaxation time (T₁, T₂) were determined. The calculated results of the longitudinal relaxation rate and transverse relaxation rate (r₁, r₂) are shown in Table 2.

TABLE 2
Synthesis of Gd-PASP and MRI characterization results

| | Gadolinium | | | | |
|---|---|---|---|---|---|
| | recovery rate | | | | |
| Sample | (%) | H₀ (T) | r₁ (mM⁻¹ s⁻¹) | r₂ (mM⁻¹ s⁻¹) | r₂/r₁ |
| Gd-PASP | 91.3% | 3.0 | 56.70 ± 1.34 | 80.23 ± 0.80 | 1.42 ± 0.05 |
| | | 7.0 | 19.60 | 76.84 | 3.92 |

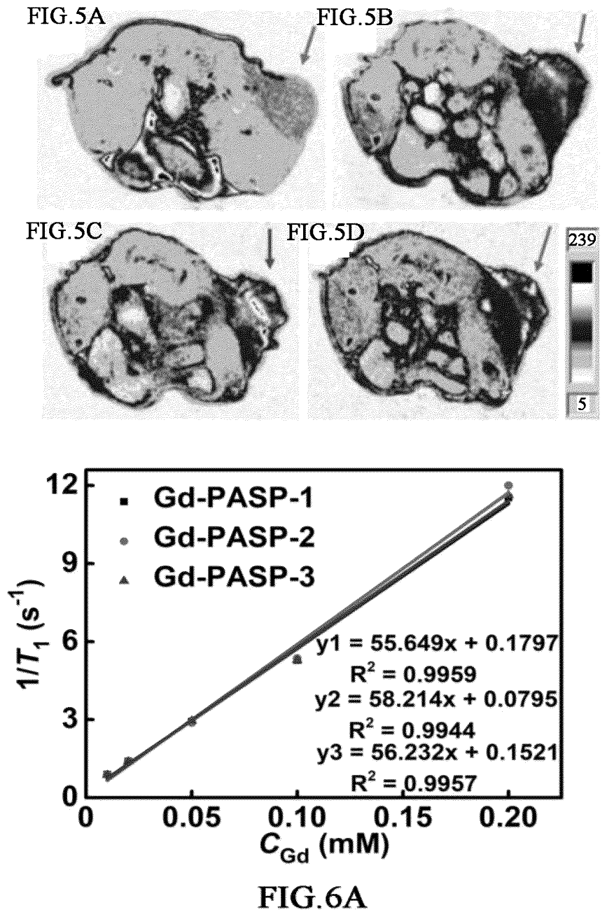

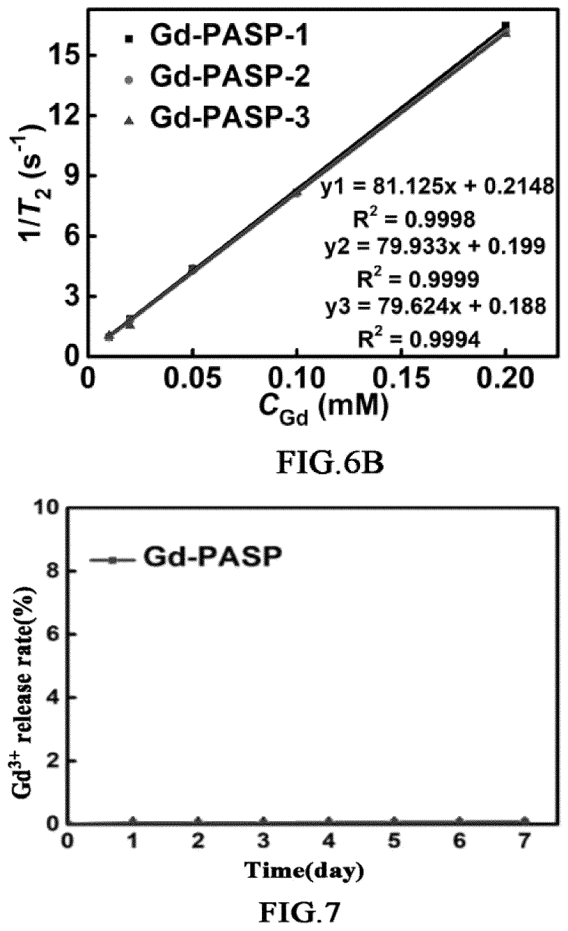

Specifically, FIG. 6A and FIG. 6B are charts showing relaxation rates of the samples Gd-PASP of Example 2 when used as an MRI contract agent (three samples Gd-PASP-1, Gd-PASP-2 and Gd-PASP-3 were subjected to a parallel test for three times), and in the relationship that 1/Tᵢ changes with the gadolinium concentration, a slope of the fitted straight line is the Tᵢ relaxation rate (i=1 or 2). It can be seen that in the 3.0 T clinical MRI scanning system (Philips, Ingenia), the r₁ value of the gadolinium chelate Gd-PASP is 56.70±1.34 mM⁻¹ s⁻¹, which is much higher than the r₁ value (r₁=4-7 mM⁻¹ s⁻¹) of the commercially available gadolinium-based contrast agent, and significantly higher than that of the highly toxic free gadolinium ion solution. In addition, the gadolinium chelate Gd-PASP also has a lower r₂/r₁ ratio, showing a better MRI contrast effect.

FIG. 7 shows a gadolinium ion release rate of the sample Gd-PASP under the condition of pH=7.4 for 1-7 days. The sample Gd-PASP hardly releases ions (<0.1%), indicating that Gd-PASP has good stability when used as the MRI contrast agent.

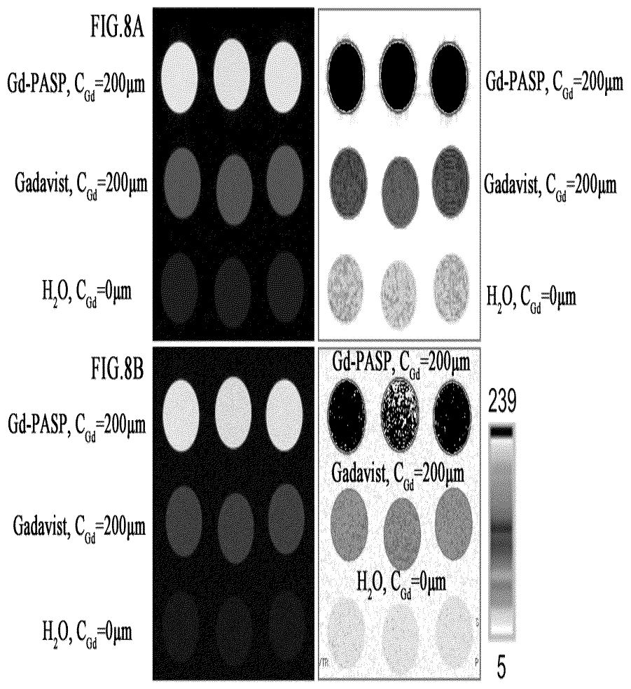

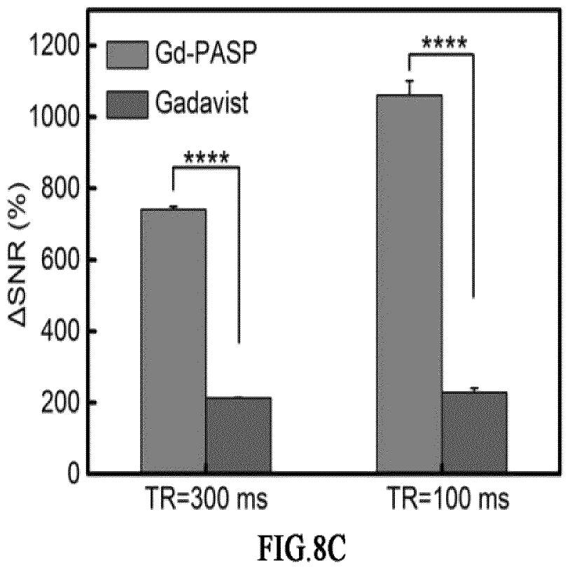

FIG. 8A, FIG. 8B and FIG. 8C show T₁-weighted MRI images of Gd-PASP, the commercially available contrast agent Gadavist and pure water and corresponding relative MRI signal intensities thereof. The magnetic field was 7.0 T (Bruker, PharmaScan 70/16 US). Wherein TR=300 ms and TE=6.5 ms in FIG. 8A, TR=100 ms and TE=6.5 ms in FIG. 8B, and FIG. 8C shows the relative MRI signal intensities of Gd-PASP, Gadavist and the pure water, ****p<0.0001. The results show that the MRI signal intensity of Gd-PASP is much higher than that of Gadavist.

### Example 3

Preparation of Gadolinium Chelate (Gd-HPMA)

40 mL of polymaleic acid (M_{w}=2000) solution with a concentration of 4.0 mg/mL was prepared, and the polymer solution was heated at 100° C. for refluxing. 0.8 mL of gadolinium nitrate solution with a concentration of 125 mM was immediately added to the reaction system, and the reaction continued for 60 minutes at 100° C. under the magnetic stirring. After being cooled to the room temperature, the solution was purified by means of membrane dialysis to obtain a product labeled as Gd-HPMA finally.

Characterization and performance tests were performed on a sample provided in Example 3. The gadolinium recovery rate of the sample prepared in Example 3 was higher, indicating that the utilization rate of raw materials was high. The sample prepared in Example 3 was prepared into at least 5 aqueous solutions with different concentrations, in vitro imaging was performed by means of the 3.0 T clinical MRI system, and a longitudinal relaxation time and a transverse relaxation time (T₁, T₂) thereof were measured. The calculated results of the longitudinal relaxation rate and transverse relaxation rate (r₁, r₂) are shown in Table 3.

TABLE 3
Synthesis of Gd-HPMA and MRI characterization results

| | Gadolinium | | | | |
|---|---|---|---|---|---|
| | recovery rate | | | | |
| Sample | (%) | H₀ (T) | r₁ (mM⁻¹ s⁻¹) | r₂ (mM⁻¹ s⁻¹) | r₂/r₁ |
| Gd-HPMA | 94.4% | 3.0 | 60.86 ± 1.65 | 86.50 ± 0.31 | 1.42 ± 0.03 |

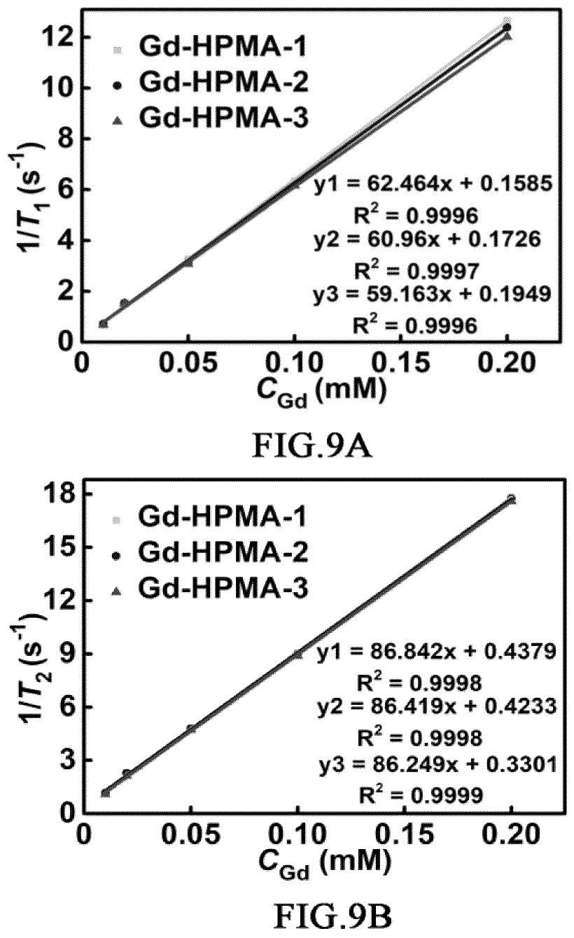

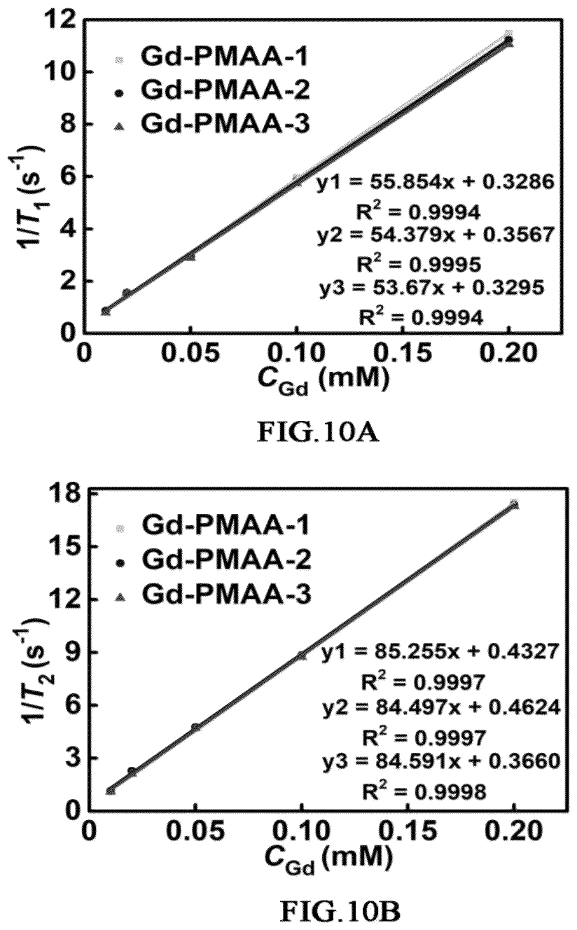

Specifically, FIG. 9 A and FIG. 9B are charts showing relaxation rates of the samples Gd-HPMA of Example 3 when used as an MRI contract agent (three samples Gd-HPMA-1, Gd-HPMA-2 and Gd-HPMA-3 were subjected to a parallel test for three times), and in the relationship that 1/Tᵢ changes with the gadolinium concentration, a slope of the fitted straight line is the Tᵢ relaxation rate (i=1 or 2). It can be seen that in the 3.0 T clinical MRI scanning system (Philips, Ingenia), the r₁ value of the gadolinium chelate Gd-HPMA is 60.86±1.65 mM⁻¹ s⁻¹, which is much higher than the r₁ value (r₁=4-7 mM⁻¹ s⁻¹) of the commercially available gadolinium-based contrast agent, and significantly higher than that of the highly toxic free gadolinium ion solution. In addition, the gadolinium chelate Gd-HPMA also has a lower r₂/r₁ ratio, showing a better MRI contrast effect.

### Example 4

Preparation of Gadolinium Chelate (Gd-PMAA)

40 mL of polymethacrylic acid (M_{w}=2000) solution with a concentration of 4.0 mg/mL was prepared, and the polymer solution was heated at 100° C. for refluxing. 0.8 mL of gadolinium nitrate solution with a concentration of 125 mM was immediately added to the reaction system, and the reaction continued for 60 minutes at 100° C. under magnetic stirring. After being cooled to the room temperature, the solution was purified by means of membrane dialysis to obtain a product labeled as Gd-PMAA finally.

Characterization and performance tests were performed on a sample provided in Example 4. The gadolinium recovery rate of the sample prepared in Example 4 was higher, indicating that the utilization rate of raw materials was high. The sample prepared in Example 4 was prepared into at least 5 aqueous solutions with different concentrations, in vitro imaging was performed by means of the 3.0 T clinical MRI system, and a longitudinal relaxation time and a transverse relaxation time (T₁, T₂) thereof were measured. The calculated results of the longitudinal relaxation rate and transverse relaxation rate (r₁, r₂) are shown in Table 4.

TABLE 4
Synthesis of Gd-PMAA and MRI characterization results

| | Gadolinium | | | | |
|---|---|---|---|---|---|
| | recovery rate | | | | |
| Sample | (%) | H₀ (T) | r₁ (mM⁻¹ s⁻¹) | r₂ (mM⁻¹ s⁻¹) | r₂/r₁ |
| Gd-PMAA | 93.6% | 3.0 | 54.63 ± 1.11 | 84.78 ± 0.41 | 1.55 ± 0.02 |

Specifically, FIG. 10A and FIG. 10B are charts showing relaxation rates of the samples Gd-PMAA of Example 4 when used as an MRI contract agent (three samples Gd-PMAA-1, Gd-PMAA-2 and Gd-PMAA-3 were subjected to a parallel test for three times), and in the relationship that 1/Tᵢ changes with the gadolinium concentration, a slope of the fitted straight line is the Tᵢ relaxation rate (i=1 or 2). It can be seen that in the 3.0 T clinical MRI scanning system (Philips, Ingenia), the r₁ value of the gadolinium chelate Gd-PMAA is 54.63±1.11 mM⁻¹ s⁻¹, which is much higher than the r₁ value (r₁=4-7 mM⁻¹ s⁻¹) of the commercially available gadolinium-based contrast agent, and significantly higher than that of the highly toxic free gadolinium ion solution. In addition, the gadolinium chelate Gd-PMAA also has a lower r₂/r₁ ratio, showing a better MRI contrast effect.

### Example 5

Preparation of Gadolinium Chelate (Gd-PEAA)

40 mL of poly(2-ethylacrylic acid) (M_{w}=2000) solution with a concentration of 4.0 mg/mL was prepared, and the polymer solution was heated at 100° C. for refluxing. 0.8 mL of gadolinium nitrate solution with a concentration of 125 mM was immediately added to the reaction system, and the reaction continued for 60 minutes at 100° C. under the magnetic stirring. After being cooled to the room temperature, the solution was purified by means of membrane dialysis to obtain a product labeled as Gd-PEAA finally.

Characterization and performance tests were performed on a sample provided in Example 5. The gadolinium recovery rate of the sample prepared in Example 5 was higher, indicating that the utilization rate of raw materials was high. The sample prepared in Example 5 was prepared into at least 5 aqueous solutions with different concentrations, in vitro imaging was performed by means of the 3.0 T clinical MRI system, and a longitudinal relaxation time and a transverse relaxation time (T₁, T₂) thereof were measured. The calculated results of the longitudinal relaxation rate and transverse relaxation rate (r₁, r₂) are shown in Table 5.

TABLE 5
Synthesis of Gd-PEAA and MRI characterization results

| | Gadolinium | | | | |
|---|---|---|---|---|---|
| | recovery rate | | | | |
| Sample | (%) | H₀ (T) | r₁ (mM⁻¹ s⁻¹) | r₂ (mM⁻¹ s⁻¹) | r₂/r₁ |
| Gd-PEAA | 93.3% | 3.0 | 53.13 ± 0.64 | 84.00 ± 1.39 | 1.58 ± 0.01 |

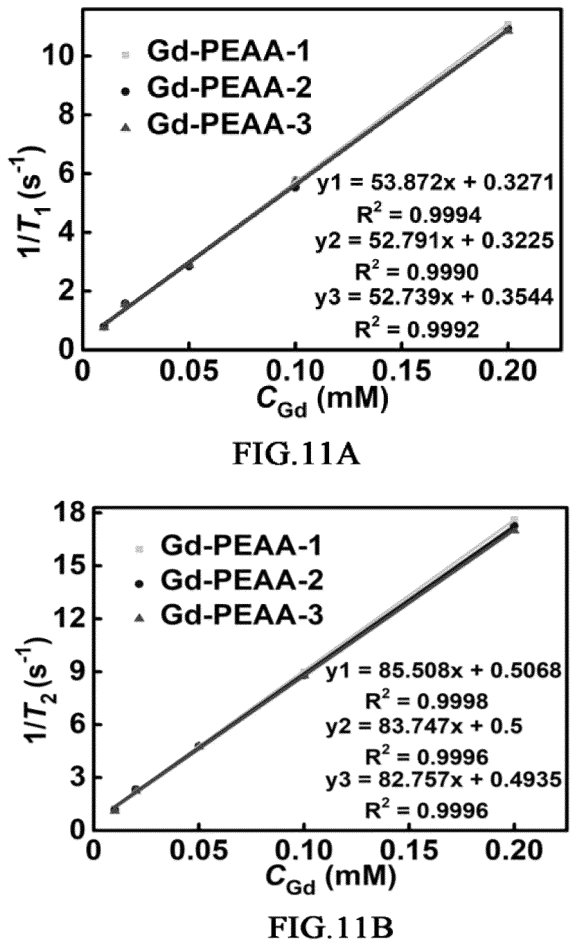

Specifically, FIG. 11A and FIG. 11B are charts showing relaxation rates of the samples Gd-PEAA of Example 5 when used as an MRI contract agent (three samples Gd-PEAA-1, Gd-PEAA-2 and Gd-PEAA-3 were subjected to a parallel test for three times), and in the relationship that 1/Tᵢ changes with the gadolinium concentration, a slope of the fitted straight line is the Tᵢ relaxation rate (i=1 or 2). It can be seen that in the 3.0 T clinical MRI scanning system (Philips, Ingenia), the r₁ value of the gadolinium chelate Gd-PEAA is 53.13±0.64 mm⁻¹ s⁻¹, which is much higher than the r₁ value (r₁=4-7 mM⁻¹ s⁻¹) of the commercially available gadolinium-based contrast agent, and significantly higher than that of the highly toxic free gadolinium ion solution. In addition, the gadolinium chelate Gd-PEAA also has a lower r₂/r₁ ratio, showing a better MRI contrast effect.

### Example 6

Preparation of Gadolinium Chelate (Gd-γ-PGA)

40 mL of polyglutamic acid (M_{w}=2000) solution with a concentration of 4.0 mg/mL was prepared, and the polymer solution was heated at 100° C. for refluxing. 0.8 mL of gadolinium nitrate solution with a concentration of 125 mM was immediately added to the reaction system, and the reaction continued for 60 minutes at 100° C. under the magnetic stirring. After being cooled to the room temperature, the solution was purified by means of membrane dialysis to obtain a product labeled as Gd-γ-PGA finally.

Characterization and performance tests were performed on a sample provided in Example 6. The gadolinium recovery rate of the sample prepared in Example 6 was higher, indicating that the utilization rate of raw materials was high. The sample prepared in Example 6 was prepared into at least 5 aqueous solutions with different concentrations, in vitro imaging was performed by means of the 3.0 T clinical MRI system, and a longitudinal relaxation time and a transverse relaxation time (T₁, T₂) thereof were measured. The calculated results of the longitudinal relaxation rate and transverse relaxation rate (r₁, r₂) are shown in Table 6.

TABLE 6
Synthesis of Gd-γ-PGA and MRI characterization results

| | Gadolinium | | | | |
|---|---|---|---|---|---|
| | recovery rate | | | | |
| Sample | (%) | H₀ (T) | r₁ (mM⁻¹ s⁻¹) | r₂ (mM⁻¹ s⁻¹) | r₂/r₁ |
| Gd-γ-PGA | 82.4% | 3.0 | 45.59 ± 1.23 | 69.39 ± 0.64 | 1.52 ± 0.03 |

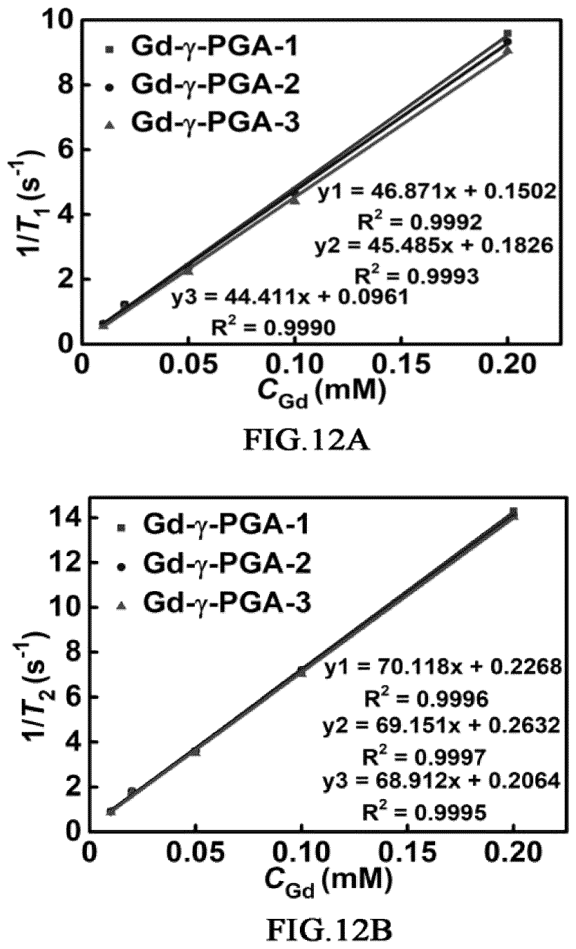

Specifically, FIG. 12A and FIG. 12B are charts showing relaxation rates of the samples Gd-γ-PGA of Example 6 when used as an MRI contract agent (three samples Gd-γ-PGA-1, Gd-γ-PGA-2 and Gd-γ-PGA-3 were subjected to a parallel test for three times), and in the relationship that 1/Tᵢ changes with the gadolinium concentration, a slope of the fitted straight line is the Tᵢ relaxation rate (i=1 or 2). It can be seen that in the 3.0 T clinical MRI scanning system (Philips, Ingenia), the r₁ value of the gadolinium chelate Gd-γ-PGA is 45.59±1.23 mM⁻¹ s⁻¹, which is much higher than the r₁ value (r₁=4-7 mM⁻¹ s⁻¹) of the commercially available gadolinium-based contrast agent, and significantly higher than that of the highly toxic free gadolinium ion solution. In addition, the gadolinium chelate Gd-γ-PGA also has a lower r₂/r₁ ratio, showing a better MRI contrast effect.

### Example 7

Preparation of Gadolinium Chelate (Gd-ε-PL)

40 mL of polylysine (M_{w}=2000) solution with a concentration of 4.0 mg/mL was prepared, and the polymer solution was heated at 100° C. for refluxing. 0.8 mL of gadolinium nitrate solution with a concentration of 125 mM was immediately added to the reaction system, and the reaction continued for 60 minutes at 100° C. under the magnetic stirring. After being cooled to the room temperature, the solution was purified by means of membrane dialysis to obtain a product labeled as Gd-ε-PL finally.

Characterization and performance tests were performed on a sample provided in Example 7. The gadolinium recovery rate of the sample prepared in Example 7 was higher, indicating that the utilization rate of raw materials was high. The sample prepared in Example 7 was prepared into at least 5 aqueous solutions with different concentrations, in vitro imaging was performed by means of the 3.0 T clinical MRI system, and a longitudinal relaxation time and a transverse relaxation time (T₁, T₂) thereof were measured. The finally calculated results of the longitudinal relaxation rate and transverse relaxation rate (r₁, r₂) are shown in Table 7.

TABLE 7
Synthesis of Gd-ε-PL and MRI characterization results

| | Gadolinium | | | | |
|---|---|---|---|---|---|
| | recovery rate | | | | |
| Sample | (%) | H₀ (T) | r₁ (mM⁻¹ s⁻¹) | r₂ (mM⁻¹ s⁻¹) | r₂/r₁ |
| Gd-ε-PL | 88.6% | 3.0 | 41.61 ± 1.20 | 67.95 ± 0.62 | 1.63 ± 0.03 |

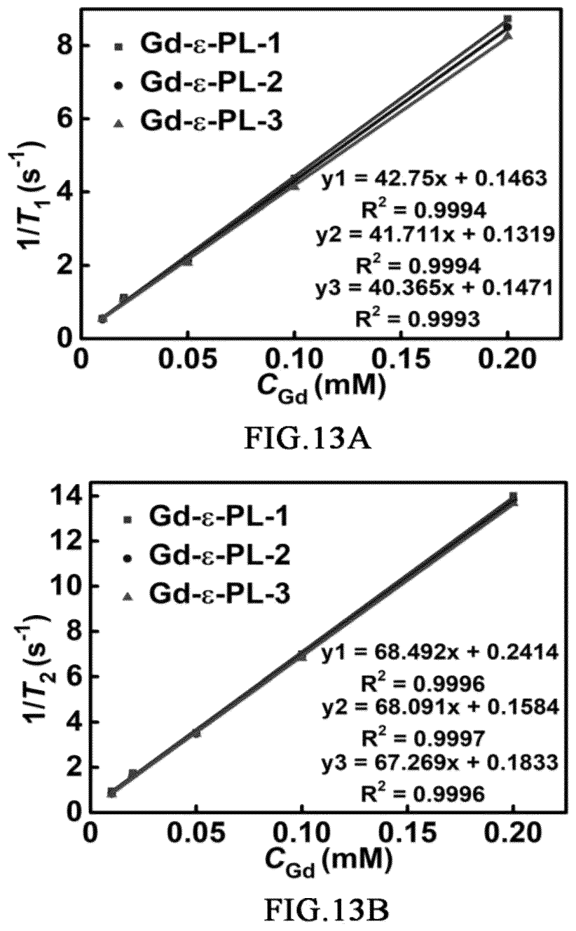

Specifically, FIG. 13A and FIG. 13B are charts showing relaxation rates of the samples Gd-ε-PL of Example 7 when used as an MRI contract agent (three samples Gd-ε-PL-1, Gd-ε-PL-2 and Gd-ε-PL-3 were subjected to a parallel test for three times), and in the relationship that 1/Tᵢ changes with the gadolinium concentration, a slope of the fitted straight line is the Tᵢ relaxation rate (i=1 or 2). It can be seen that in the 3.0 T clinical MRI scanning system (Philips, Ingenia), the r₁ value of the gadolinium chelate Gd-ε-PL is 41.61±1.20 mM⁻¹ s⁻¹, which is much higher than the r₁ value (r₁=4-7 mM⁻¹ s⁻¹) of the commercially available gadolinium-based contrast agent, and significantly higher than that of the highly toxic free gadolinium ion solution. In addition, the gadolinium chelate Gd-ε-PL also has a lower r₂/r₁ ratio, showing a better MRI contrast effect.

### Example 8

Preparation of Gadolinium Chelate (Gd-PLR)

40 mL of polyarginine (M_{w}=2000) solution with a concentration of 4.0 mg/mL was prepared, and the polymer solution was heated at 100° C. for refluxing. 0.8 mL of gadolinium nitrate solution with a concentration of 125 mM was immediately added to the reaction system, and the reaction continued for 60 minutes at 100° C. under the magnetic stirring. After being cooled to the room temperature, the solution was purified by means of membrane dialysis to obtain a product labeled as Gd-PLR finally.

Characterization and performance tests were performed on a sample provided in Example 8. The gadolinium recovery rate of the sample prepared in Example 8 was higher, indicating that the utilization rate of raw materials was high. The sample prepared in Example 8 was prepared into at least 5 aqueous solutions with different concentrations, in vitro imaging was performed by means of the 3.0 T clinical MRI system, and a longitudinal relaxation time and a transverse relaxation time (T₁, T₂) thereof were measured. The finally calculated results of the longitudinal relaxation rate and transverse relaxation rate (r₁, r₂) are shown in Table 8.

TABLE 8
Synthesis of Gd-PLR and MRI characterization results

| | Gadolinium | | | | |
|---|---|---|---|---|---|
| | recovery rate | | | | |
| Sample | (%) | H₀ (T) | r₁ (mM⁻¹ s⁻¹) | r₂ (mM⁻¹ s⁻¹) | r₂/r₁ |
| Gd-PLR | 87.8% | 3.0 | 38.90 ± 0.78 | 67.54 ± 0.80 | 1.74 ± 0.02 |

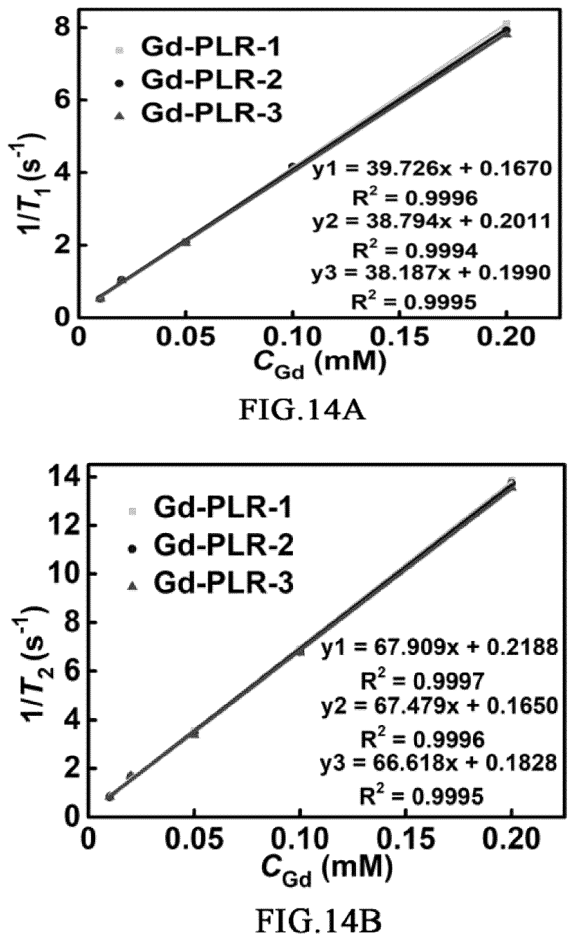

Specifically, FIG. 14A and FIG. 14B are charts showing relaxation rates of the samples Gd-PLR of Example 8 when used as an MRI contract agent (three samples Gd-PLR-1, Gd-PLR-2 and Gd-PLR-3 were subjected to a parallel test for three times), and in the relationship that 1/Tᵢ changes with the gadolinium concentration, a slope of the fitted straight line is the Tᵢ relaxation rate (i=1 or 2). It can be seen that in the 3.0 T clinical MRI scanning system (Philips, Ingenia), the r₁ value of the gadolinium chelate Gd-PLR is 38.90±0.78 mM⁻¹ s⁻¹, which is much higher than the r₁ value (r₁=4-7 mM⁻¹ s⁻¹) of the commercially available gadolinium-based contrast agent, and significantly higher than that of the highly toxic free gadolinium ion solution. In addition, the gadolinium chelate Gd-PLR also has a lower r₂/r₁ ratio, showing a better MRI contrast effect.

### Example 9

Preparation of Gadolinium Chelate (Gd-PLH)

40 mL of polyhistidine (M_{w}=2000) solution with a concentration of 4.0 mg/mL was prepared, and the polymer solution was heated at 100° C. for refluxing. 0.8 mL of gadolinium nitrate solution with a concentration of 125 mM was immediately added to the reaction system, and the reaction continued for 60 minutes at 100° C. under magnetic stirring. After being cooled to the room temperature, the solution was purified by means of membrane dialysis to obtain a product labeled as Gd-PLH finally.

Characterization and performance tests were performed on a sample provided in Example 9. The gadolinium recovery rate of the sample prepared in Example 9 was higher, indicating that the utilization rate of raw materials was high. The sample prepared in Example 9 was prepared into at least 5 aqueous solutions with different concentrations, in vitro imaging was performed by means of the 3.0 T clinical MRI system, and a longitudinal relaxation time and a transverse relaxation time (T₁, T₂) thereof were measured. The finally calculated results of the longitudinal relaxation rate and transverse relaxation rate (r₁, r₂) are shown in Table 9.

TABLE 9
Synthesis of Gd-PLH and MRI characterization results

| | Gadolinium | | | | |
|---|---|---|---|---|---|
| | recovery rate | | | | |
| Sample | (%) | H₀ (T) | r₁ (mM⁻¹ s⁻¹) | r₂ (mM⁻¹ s⁻¹) | r₂/r₁ |
| Gd-PLH | 90.2% | 3.0 | 45.46 ± 0.95 | 68.65 ± 0.39 | 1.51 ± 0.02 |

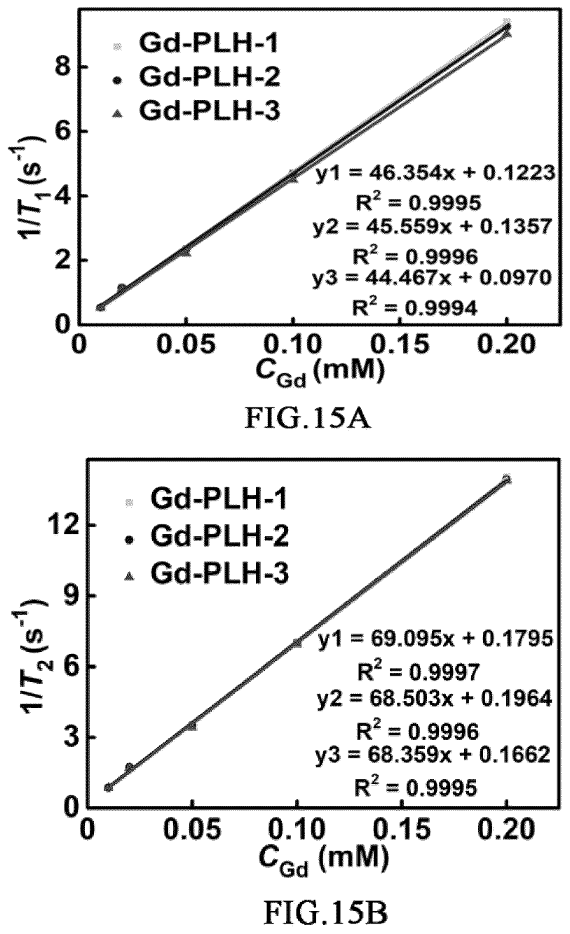

Specifically, FIG. 15A and FIG. 15B are charts showing relaxation rates of the samples Gd-PLH of Example 9 when used as an MRI contract agent (three samples Gd-PLH-1, Gd-PLH-2 and Gd-PLH-3 were subjected to a parallel test for three times), and in the relationship that 1/Tᵢ changes with the gadolinium concentration, a slope of the fitted straight line is the Tᵢ relaxation rate (i=1 or 2). It can be seen that in the 3.0 T clinical MRI scanning system (Philips, Ingenia), the r₁ value of the gadolinium chelate Gd-PLH is 45.46±0.95 mM⁻¹ s⁻¹, which is much higher than the r₁ value (r₁=4-7 mM⁻¹ s⁻¹) of the commercially available gadolinium-based contrast agent, and significantly higher than that of the highly toxic free gadolinium ion solution. In addition, the gadolinium chelate Gd-PLH also has a lower r₂/r₁ ratio, showing a better MRI contrast effect.

### Example 10

Preparation of Gadolinium Chelate (Gd-PEI)

40 mL of polyethyleneimine (M_{w}=2000) solution with a concentration of 4.0 mg/mL was prepared, and the polymer solution was heated at 100° C. for refluxing. 0.8 mL of gadolinium nitrate solution with a concentration of 125 mM was immediately added to the reaction system, and the reaction continued for 60 minutes at 100° C. under the magnetic stirring. After being cooled to the room temperature, the solution was purified by means of membrane dialysis to obtain a product labeled as Gd-PEI finally.

Characterization and performance tests were performed on a sample provided in Example 10. The gadolinium recovery rate of the sample prepared in Example 10 was higher than 90%, indicating that the utilization rate of raw materials was high. The sample prepared in Example 10 was prepared into at least 5 aqueous solutions with different concentrations, in vitro imaging was performed by means of the 3.0 T clinical MRI system, and a longitudinal relaxation time and a transverse relaxation time (T₁, T₂) thereof were measured. The calculated results of the longitudinal relaxation rate and transverse relaxation rate (r₁, r₂) are shown in Table 10.

TABLE 10
Synthesis of Gd-PEI and MRI characterization results

| | Gadolinium | | | | |
|---|---|---|---|---|---|
| | recovery rate | | | | |
| Sample | (%) | H₀ (T) | r₁ (mM⁻¹ s⁻¹) | r₂ (mM⁻¹ s⁻¹) | r₂/r₁ |
| Gd-PEI | 90.5% | 3.0 | 41.09 ± 0.77 | 64.51 ± 0.94 | 1.57 ± 0.04 |

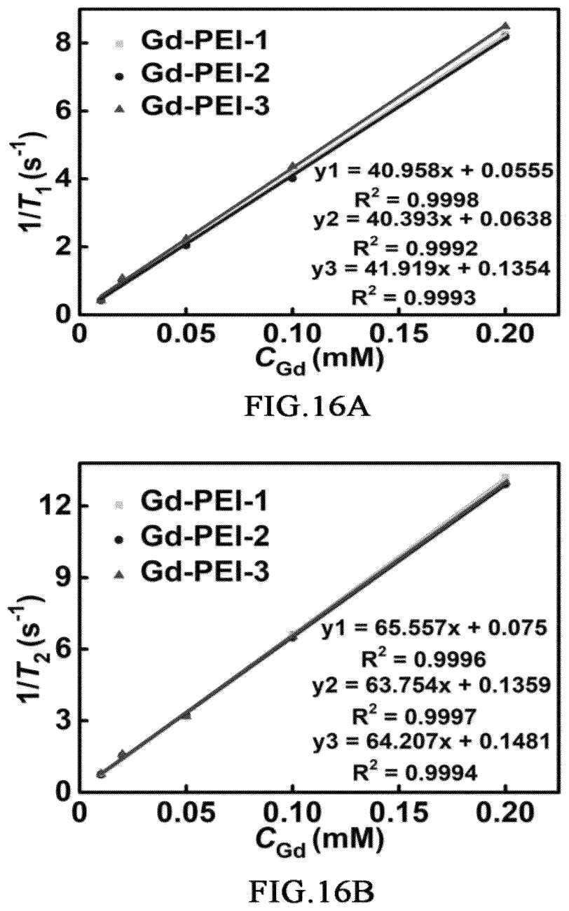

Specifically, FIG. 16A and FIG. 16B are charts showing relaxation rates of the samples Gd-PEI of Example 10 when used as an MRI contract agent (three samples Gd-PEI-1, Gd-PEI-2 and Gd-PEI-3 were subjected to a parallel test for three times), and in the relationship that 1/Tᵢ changes with the gadolinium concentration, a slope of the fitted straight line is the Tᵢ relaxation rate (i=1 or 2). It can be seen that in the 3.0 T clinical MRI scanning system (Philips, Ingenia), the r₁ value of the gadolinium chelate Gd-PEI is 41.09±0.77 mM⁻¹ s⁻¹, which is much higher than the r₁ value (r₁=4-7 mM⁻¹ s⁻¹) of the commercially available gadolinium-based contrast agent, and significantly higher than that of the highly toxic free gadolinium ion solution. In addition, the gadolinium chelate Gd-PEI also has a lower r₂/r₁ ratio, showing a better MRI contrast effect.

### Example 11

Preparation of Gadolinium Chelate (Gd-PSer)

40 mL of polyserine (M_{w}=2000) solution with a concentration of 4.0 mg/mL was prepared, and the polymer solution was heated at 100° C. for refluxing. 0.8 mL of gadolinium nitrate solution with a concentration of 125 mM was immediately added to the reaction system, and the reaction continued for 60 minutes at 100° C. under the magnetic stirring. After being cooled to the room temperature, the solution was purified by means of membrane dialysis to obtain a product labeled as Gd-PSer finally.

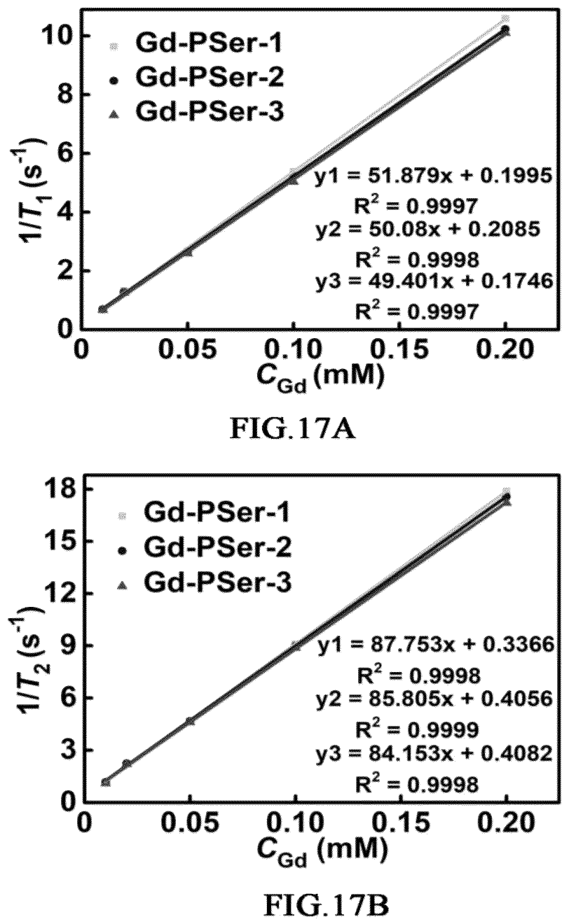

Characterization and performance tests were performed on a sample provided in Example 11. The gadolinium recovery rate of the sample prepared in Example 11 was 88.3%, indicating that the utilization rate of raw materials was higher. The sample prepared in Example 11 was prepared into at least 5 aqueous solutions with different concentrations, in vitro imaging was performed by means of the 3.0 T clinical MRI system (Philips, Ingenia), and a longitudinal relaxation time and a transverse relaxation time (T₁, T₂) thereof were measured (three samples Gd-PSer-1, Gd-PSer-2 and Gd-PSer-3 were subjected to a parallel test for three times). As shown in FIG. 17A and FIG. 17B, the relationship of 1/Tᵢ changing with the gadolinium concentration is drawn, and a slope of the fitted straight line is the Tᵢ relaxation rate (i=1 or 2). It can be seen that in the 3.0 T MRI scanning system, the r₁ value of the gadolinium chelate Gd-PSer is 50.45±1.28 mM⁻¹ s⁻¹, the r₂ value is 85.90±1.80 mM⁻¹ s⁻¹, and r₂/r₁ is 1.70±0.01. The r₁ value is much higher than the r₁ value (r₁=4-7 mM⁻¹ s⁻¹) of the commercially available gadolinium-based contrast agent, and significantly higher than that of the highly toxic free gadolinium ion solution. In addition, the gadolinium chelate Gd-PSer also has a lower r₂/r₁ ratio, showing a better MRI contrast effect.

### Example 12

Preparation of Gadolinium Chelate (Gd-PThr)

40 mL of polythreonine (M_{w}=2000) solution with a concentration of 4.0 mg/mL was prepared, and the polymer solution was heated at 100° C. for refluxing. 0.8 mL of gadolinium nitrate solution with a concentration of 125 mM was immediately added to the reaction system, and the reaction continued for 60 minutes at 100° C. under the magnetic stirring. After being cooled to the room temperature, the solution was purified by means of membrane dialysis to obtain a product labeled as Gd-PThr finally.

Characterization and performance tests were performed on a sample provided in Example 12. The gadolinium recovery rate of the sample prepared in Example 12 was 88.3%, indicating that the utilization rate of raw materials was higher. The sample prepared in Example 12 was prepared into at least 5 aqueous solutions with different concentrations, in vitro imaging was performed by means of the 3.0 T clinical MRI system (Philips, Ingenia), and a longitudinal relaxation time and a transverse relaxation time (T₁, T₂) thereof were measured (three samples Gd-PThr-1, Gd-PThr-2 and Gd-PThr-3 were subjected to a parallel test for three times). As shown in FIG. 18A and FIG. 18B, the relationship of 1/Tᵢ changing with the gadolinium concentration is drawn, and a slope of the fitted straight line is the Tᵢ relaxation rate (i=1 or 2). It can be seen that in the 3.0 T MRI scanning system, the r₁ value of the gadolinium chelate Gd-PThr is 48.21±0.96 mM⁻¹ s⁻¹, the r₂ value is 81.42±1.68 mM⁻¹ s⁻¹, and r₂/r₁ is 1.69±0.00. The r₁ value is much higher than the r₁ value (r₁=4-7 mM⁻¹ s⁻¹) of the commercially available gadolinium-based contrast agent, and significantly higher than that of the highly toxic free gadolinium ion solution. In addition, the gadolinium chelate Gd-PThr also has a lower r₂/r₁ ratio, showing a better MRI contrast effect.

### Example 13

Preparation of Gadolinium Chelate (Gd-PTyr)

40 mL of polytyrosine (M_{w}=2000) solution with a concentration of 4.0 mg/mL was prepared, and the polymer solution was heated at 100° C. for refluxing. 0.8 mL of gadolinium nitrate solution with a concentration of 125 mM was immediately added to the reaction system, and the reaction continued for 60 minutes at 100° C. under the magnetic stirring. After being cooled to the room temperature, the solution was purified by means of membrane dialysis to obtain a product labeled as Gd-PTyr finally.

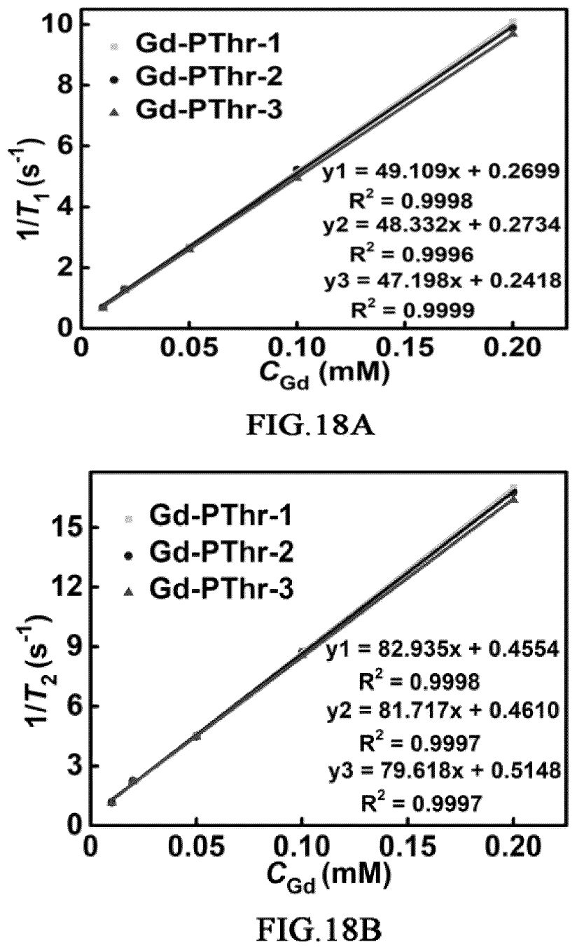

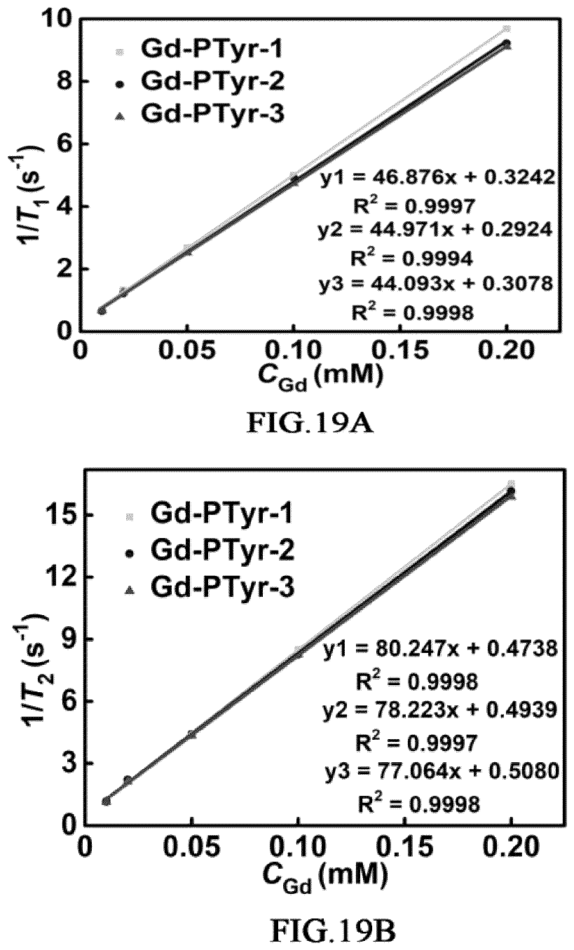

Characterization and performance tests were performed on a sample provided in Example 13. The gadolinium recovery rate of the sample prepared in Example 13 was 88.3%, indicating that the utilization rate of raw materials was higher. The sample prepared in Example 13 was prepared into at least 5 aqueous solutions with different concentrations, in vitro imaging was performed by means of the 3.0 T clinical MRI system (Philips, Ingenia), and a longitudinal relaxation time and a transverse relaxation time (T₁, T₂) thereof were measured (three samples Gd-PTyr-1, Gd-PTyr-2 and Gd-PTyr-3 were subjected to a parallel test for three times). As shown in FIG. 19A and FIG. 19B, the relationship of 1/Tᵢ changing with the gadolinium concentration is drawn, and a slope of the fitted straight line is the Tᵢ relaxation rate (i=1 or 2). It can be seen that in the 3.0 T MRI scanning system, the r₁ value of the gadolinium chelate Gd-PTyr is 45.31±1.42 mM⁻¹ s⁻¹, the r₂ value is 78.51±1.61 mM⁻¹ s⁻¹, and r₂/r₁ is 1.73±0.02. The r₁ value is much higher than the r₁ value (r₁=4-7 mM⁻¹ s⁻¹) of the commercially available gadolinium-based contrast agent, and significantly higher than that of the highly toxic free gadolinium ion solution. In addition, the gadolinium chelate Gd-PTyr also has a lower r₂/r₁ ratio, showing a better MRI contrast effect.

### Example 14

Preparation of Gadolinium Chelate (Gd-PVA)

40 mL of polyvinyl alcohol (M_{w}=2000) solution with a concentration of 4.0 mg/mL was prepared, and the polymer solution was heated at 100° C. for refluxing. 0.8 mL of gadolinium nitrate solution with a concentration of 125 mM was immediately added to the reaction system, and the reaction continued for 60 minutes at 100° C. under magnetic stirring. After being cooled to the room temperature, the solution was purified by means of membrane dialysis to obtain a product labeled as Gd-PVA finally.

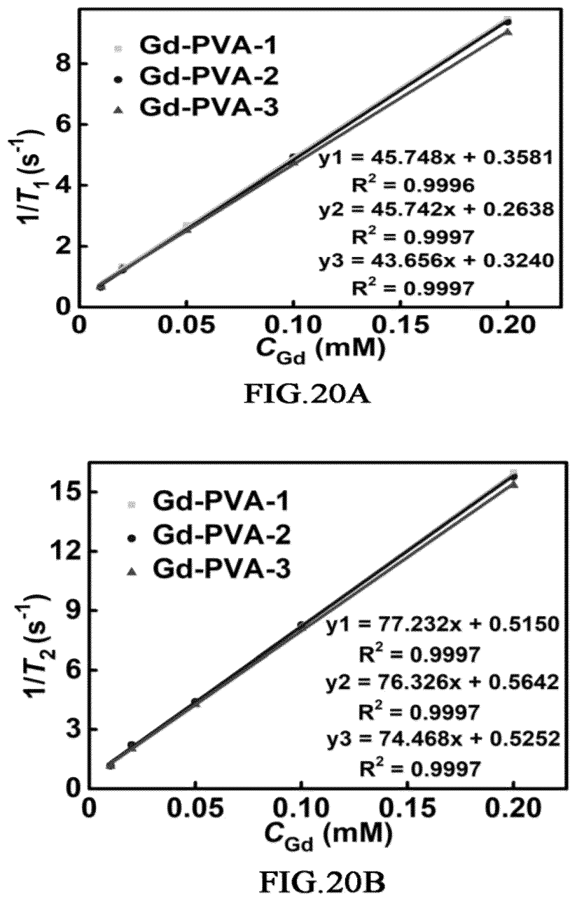

Characterization and performance tests were performed on a sample provided in Example 14. The gadolinium recovery rate of the sample prepared in Example 14 was 88.3%, indicating that the utilization rate of raw materials was higher. The sample prepared in Example 14 was prepared into at least 5 aqueous solutions with different concentrations, in vitro imaging was performed by means of the 3.0 T clinical MRI system (Philips, Ingenia), and a longitudinal relaxation time and a transverse relaxation time (T₁, T₂) thereof were measured (three samples Gd-PVA-1, Gd-PVA-2 and Gd-PVA-3 were subjected to a parallel test for three times). As shown in FIG. 20A and FIG. 20B, the relationship of 1/Tᵢ changing with the gadolinium concentration is drawn, and a slope of the fitted straight line is the Tᵢ relaxation rate (i=1 or 2). It can be seen that in the 3.0 T MRI scanning system, the r₁ value of the gadolinium chelate Gd-PVA is 45.05±1.21 mM⁻¹ s⁻¹, the r₂ value is 76.01±1.41 mM⁻¹ s⁻¹, and r₂/r₁ is 1.69±0.02. The r₁ value is much higher than the r₁ value (r₁=4-7 mM⁻¹ s⁻¹) of the commercially available gadolinium-based contrast agent, and significantly higher than that of the highly toxic free gadolinium ion solution. In addition, the gadolinium chelate Gd-PVA also has a lower r₂/r₁ ratio, showing a better MRI contrast effect.

### Example 15

Preparation of Gadolinium Chelate (Gd-PLA)

40 mL of polylactic acid (M_{w}=2000) solution with a concentration of 4.0 mg/mL was prepared, and the polymer solution was heated at 100° C. for refluxing. 0.8 mL of gadolinium nitrate solution with a concentration of 125 mM was immediately added to the reaction system, and the reaction continued for 60 minutes at 100° C. under the magnetic stirring. After being cooled to the room temperature, the solution was purified by means of membrane dialysis to obtain a product labeled as Gd-PLA finally.

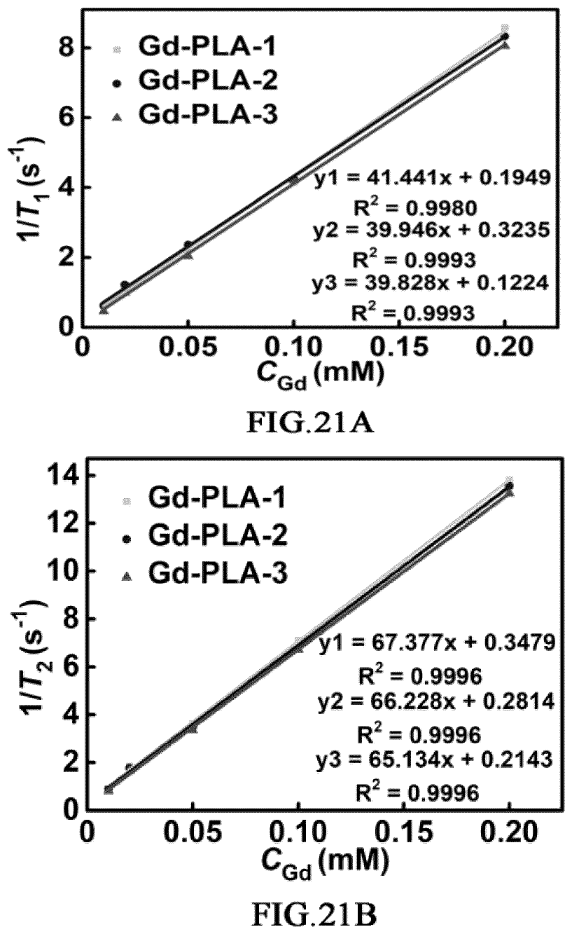

Characterization and performance tests were performed on a sample provided in Example 15. The gadolinium recovery rate of the sample prepared in Example 15 was 89.3%, indicating that the utilization rate of raw materials was higher. The sample prepared in Example 15 was prepared into at least 5 aqueous solutions with different concentrations, in vitro imaging was performed by means of the 3.0 T clinical MRI system (Philips, Ingenia), and a longitudinal relaxation time and a transverse relaxation time (T₁, T₂) thereof were measured (three samples Gd-PLA-1, Gd-PLA-2 and Gd-PLA-3 were subjected to a parallel test for three times). As shown in FIG. 21A and FIG. 21B, the relationship of 1/Tᵢ changing with the gadolinium concentration is drawn, and a slope of the fitted straight line is the Tᵢ relaxation rate (i=1 or 2). It can be seen that in the 3.0 T MRI scanning system, the r₁ value of the gadolinium chelate Gd-PLA is 40.41±0.90 mM⁻¹ s⁻¹, the r₂ value is 66.25±1.12 mM⁻¹ s⁻¹, and r₂/r₁ is 1.64±0.02. The r₁ value is much higher than the r₁ value (r₁=4-7 mM⁻¹ s⁻¹) of the commercially available gadolinium-based contrast agent, and significantly higher than that of the highly toxic free gadolinium ion solution. In addition, the gadolinium chelate Gd-PLA also has a lower r₂/r₁ ratio, showing a better MRI contrast effect.

### Example 16

Preparation of Gadolinium Chelate (Gd-PGA)

40 mL of polyglycolic acid (M_{w}=2000) solution with a concentration of 4.0 mg/mL was prepared, and the polymer solution was heated at 100° C. for refluxing. 0.8 mL of gadolinium nitrate solution with a concentration of 125 mM was immediately added to the reaction system, and the reaction continued for 60 minutes at 100° C. under the magnetic stirring. After being cooled to the room temperature, the solution was purified by means of membrane dialysis to obtain a product labeled as Gd-PGA finally.

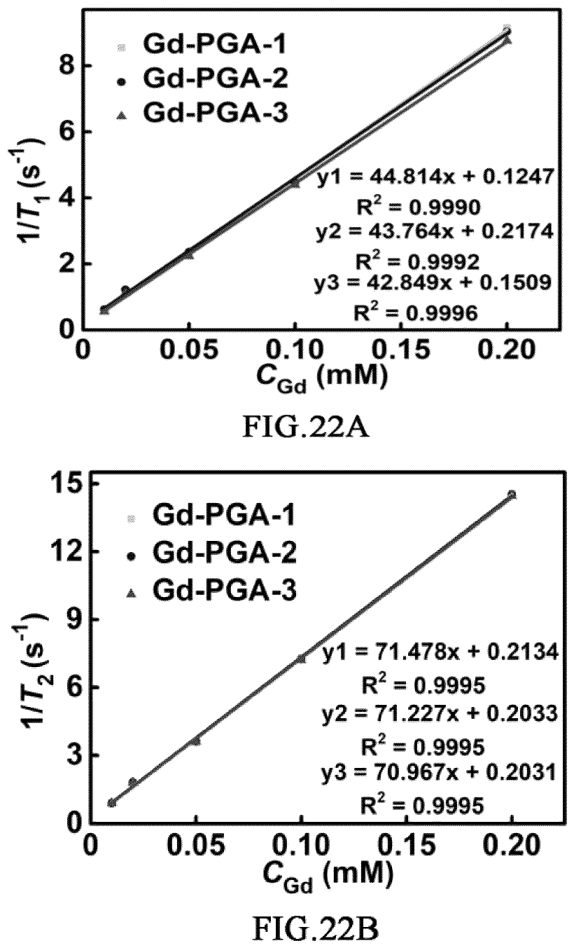

Characterization and performance tests were performed on a sample provided in Example 16. The gadolinium recovery rate of the sample prepared in Example 16 was 90.9%, indicating that the utilization rate of raw materials was higher. The sample prepared in Example 16 was prepared into at least 5 aqueous solutions with different concentrations, in vitro imaging was performed by means of the 3.0 T clinical MRI system (Philips, Ingenia), and a longitudinal relaxation time and a transverse relaxation time (T₁, T₂) thereof were measured (three samples Gd-PGA-1, Gd-PGA-2 and Gd-PGA-3 were subjected to a parallel test for three times). As shown in FIG. 22A and FIG. 22B, the relationship of 1/Tᵢ changing with the gadolinium concentration is drawn, and a slope of the fitted straight line is the Tᵢ relaxation rate (i=1 or 2). It can be seen that in the 3.0 T MRI scanning system, the r₁ value of the gadolinium chelate Gd-PGA is 43.81±0.98 mM⁻¹ s⁻¹, the r₂ value is 71.22±0.26 mM⁻¹ s⁻¹, and r₂/r₁ is 1.63±0.03. The r₁ value is much higher than the r₁ value (r₁=4-7 mM⁻¹ s⁻¹) of the commercially available gadolinium-based contrast agent, and significantly higher than that of the highly toxic free gadolinium ion solution. In addition, the gadolinium chelate Gd-PGA also has a lower r₂/r₁ ratio, showing a better MRI contrast effect.

### Example 17

Preparation of Gadolinium Chelate (Gd-PCL)

40 mL of polycaprolactone (M_{w}=2000) solution with a concentration of 4.0 mg/mL was prepared, and the polymer solution was heated at 100° C. for refluxing. 0.8 mL of gadolinium nitrate solution with a concentration of 125 mM was immediately added to the reaction system, and the reaction continued for 60 minutes at 100° C. under the magnetic stirring. After being cooled to the room temperature, the solution was purified by means of membrane dialysis to obtain a product labeled as Gd-PCL finally.

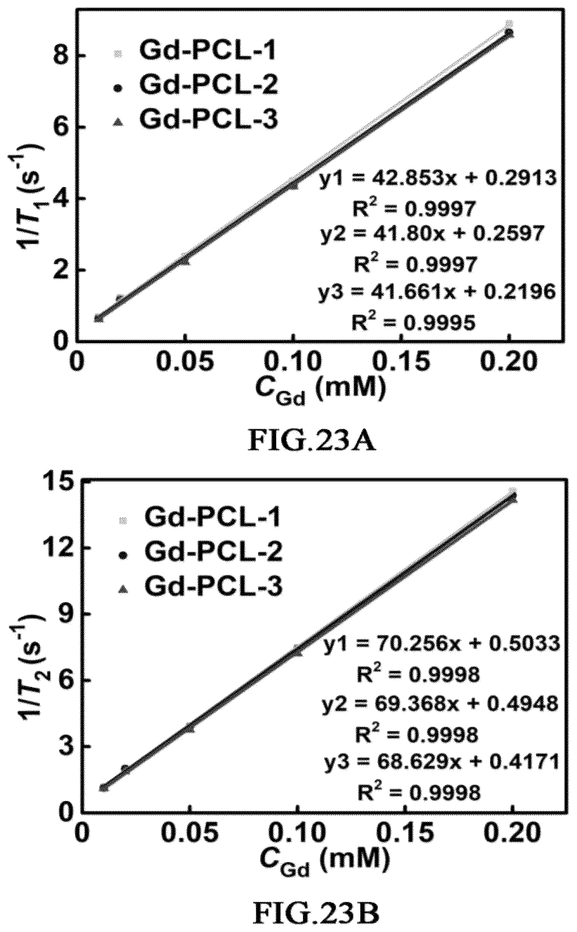

Characterization and performance tests were performed on a sample provided in Example 17. The gadolinium recovery rate of the sample prepared in Example 17 was 86.6%, indicating that the utilization rate of raw materials was higher. The sample prepared in Example 17 was prepared into at least 5 aqueous solutions with different concentrations, in vitro imaging was performed by means of the 3.0 T clinical MRI system (Philips, Ingenia), and a longitudinal relaxation time and a transverse relaxation time (T₁, T₂) thereof were measured (three samples Gd-PCL-1, Gd-PCL-2 and Gd-PCL-3 were subjected to a parallel test for three times). As shown in FIG. 23A and FIG. 23B, the relationship of 1/Tᵢ changing with the gadolinium concentration is drawn, and a slope of the fitted straight line is the Tᵢ relaxation rate (i=1 or 2). It can be seen that in the 3.0 T MRI scanning system, the r₁ value of the gadolinium chelate Gd-PCL is 42.10±0.65 mM⁻¹ s⁻¹, the r₂ value is 69.42±0.81 mM⁻¹ s⁻¹, and r₂/r₁ is 1.65±0.01. The r₁ value is much higher than the r₁ value (r₁=4-7 mM⁻¹ s⁻¹) of the commercially available gadolinium-based contrast agent, and significantly higher than that of the highly toxic free gadolinium ion solution. In addition, the gadolinium chelate Gd-PCL also has a lower r₂/r₁ ratio, showing a better MRI contrast effect.

### Example 18

Preparation of Gadolinium Chelate (Gd-PHEMA)

40 mL of poly(2-hydroxyethyl methacrylate) (M_{w}=2000) solution with a concentration of 4.0 mg/mL was prepared, and the polymer solution was heated at 100° C. for refluxing. 0.8 mL of gadolinium nitrate solution with a concentration of 125 mM was immediately added to the reaction system, and the reaction continued for 60 minutes at 100° C. under the magnetic stirring. After being cooled to the room temperature, the solution was purified by means of membrane dialysis to obtain a product labeled as Gd-PHEMA finally.

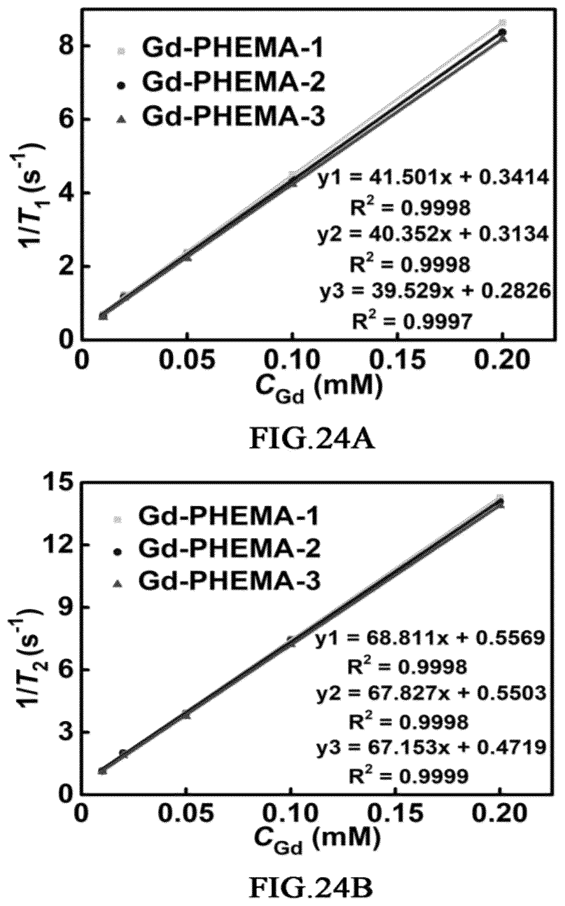

Characterization and performance tests were performed on a sample provided in Example 18. The gadolinium recovery rate of the sample prepared in Example 18 was 87.2%, indicating that the utilization rate of raw materials was higher. The sample prepared in Example 18 was prepared into at least 5 aqueous solutions with different concentrations, in vitro imaging was performed by means of the 3.0 T clinical MRI system (Philips, Ingenia), and a longitudinal relaxation time and a transverse relaxation time (T₁, T₂) thereof were measured (three samples Gd-PHEMA-1, Gd-PHEMA-2 and Gd-PHEMA-3 were subjected to a parallel test for three times). As shown in FIG. 24A and FIG. 24B, the relationship of 1/Tᵢ changing with the gadolinium concentration is drawn, and a slope of the fitted straight line is the Tᵢ relaxation rate (i=1 or 2). It can be seen that in the 3.0 T MRI scanning system, the r₁ value of the gadolinium chelate Gd-PHEMA is 40.46±0.99 mM⁻¹ s⁻¹, the r₂ value is 67.93±0.83 mM⁻¹ s⁻¹, and r₂/r₁ is 1.68±0.02. The r₁ value is much higher than the r₁ value (r₁=4-7 mM⁻¹ s⁻¹) of the commercially available gadolinium-based contrast agent, and significantly higher than that of the highly toxic free gadolinium ion solution. In addition, the gadolinium chelate Gd-PHEMA also has a lower r₂/r₁ ratio, showing a better MRI contrast effect.

### Example 19

Preparation of Gadolinium Chelate (Gd-PEG)

40 mL of polyethylene glycol (M_{w}=2000) solution with a concentration of 4.0 mg/mL was prepared, and the polymer solution was heated at 100° C. for refluxing. 0.8 mL of gadolinium nitrate solution with a concentration of 125 mM was immediately added to the reaction system, and the reaction continued for 60 minutes at 100° C. under the magnetic stirring. After being cooled to the room temperature, the solution was purified by means of membrane dialysis to obtain a product labeled as Gd-PEG finally.

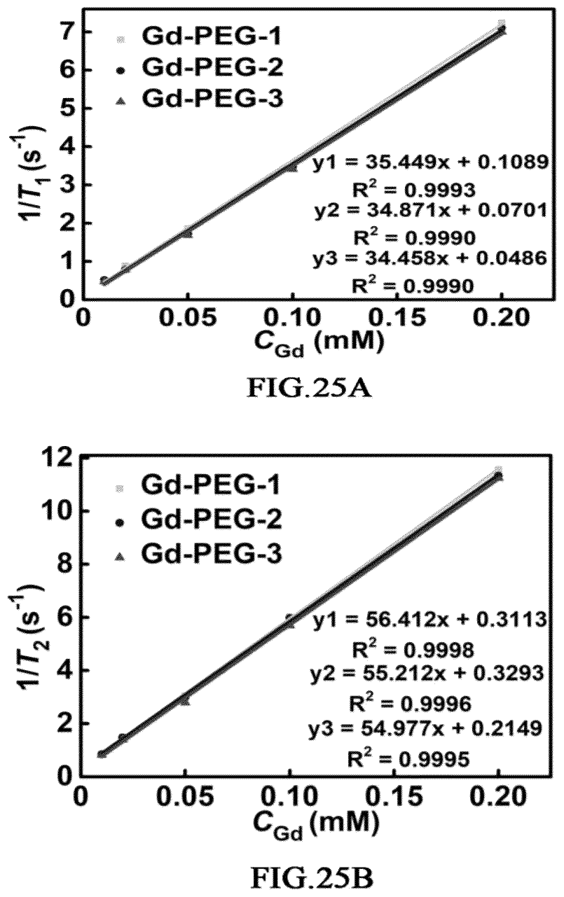

Characterization and performance tests were performed on a sample provided in Example 19. The gadolinium recovery rate of the sample prepared in Example 19 was 94.5%, indicating that the utilization rate of raw materials was high. The sample prepared in Example 19 was prepared into at least 5 aqueous solutions with different concentrations, in vitro imaging was performed by means of the 3.0 T clinical MRI system (Philips, Ingenia), and a longitudinal relaxation time and a transverse relaxation time (T₁, T₂) thereof were measured (three samples Gd-PEG-1, Gd-PEG-2 and Gd-PEG-3 were subjected to a parallel test for three times). As shown in FIG. 25A and FIG. 25B, the relationship of 1/Tᵢ changing with the gadolinium concentration is drawn, and a slope of the fitted straight line is the Tᵢ relaxation rate (i=1 or 2). It can be seen that in the 3.0 T MRI scanning system, the r₁ value of the gadolinium chelate Gd-PEG is 34.93±0.50 mM⁻¹ s⁻¹, the r₂ value is 55.53±0.77 mM⁻¹ s⁻¹, and r₂/r₁ is 1.59±0.01. The r₁ value is much higher than the r₁ value (r₁=4-7 mM⁻¹ s⁻¹) of the commercially available gadolinium-based contrast agent, and significantly higher than that of the highly toxic free gadolinium ion solution. In addition, the gadolinium chelate Gd-PEG also has a lower r₂/r₁ ratio, showing a better MRI contrast effect.

### Example 20

Preparation of Gadolinium Chelate (Gd-PPG)

40 mL of polypropylene glycol (M_{w}=2000) solution with a concentration of 4.0 mg/mL was prepared, and the polymer solution was heated at 100° C. for refluxing. 0.8 mL of gadolinium nitrate solution with a concentration of 125 mM was immediately added to the reaction system, and the reaction continued for 60 minutes at 100° C. under the magnetic stirring. After being cooled to the room temperature, the solution was purified by means of membrane dialysis to obtain a product labeled as Gd-PPG finally.

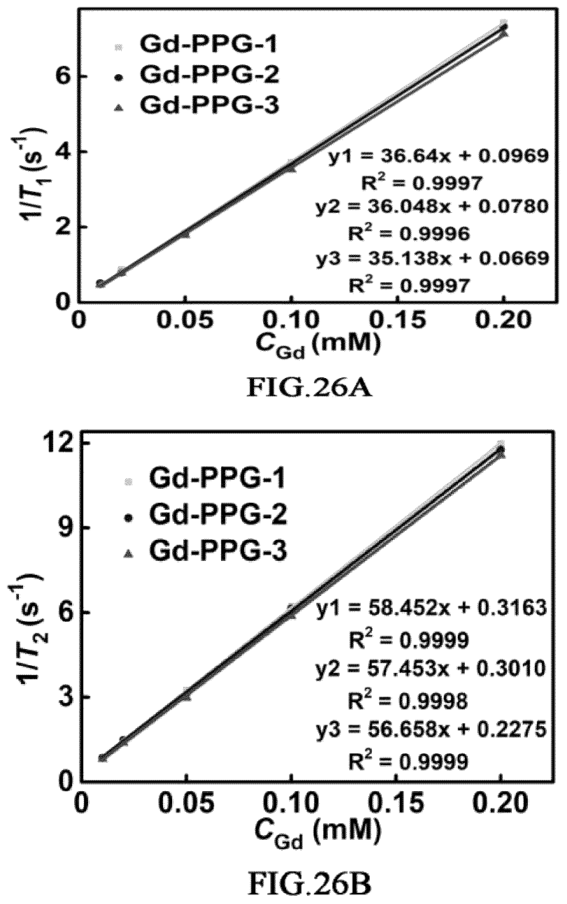

Characterization and performance tests were performed on a sample provided in Example 20. The gadolinium recovery rate of the sample prepared in Example 20 was 89.2%, indicating that the utilization rate of raw materials was higher. The sample prepared in Example 20 was prepared into at least 5 aqueous solutions with different concentrations, in vitro imaging was performed by means of the 3.0 T clinical MRI system (Philips, Ingenia), and a longitudinal relaxation time and a transverse relaxation time (T₁, T₂) thereof were measured (three samples Gd-PPG-1, Gd-PPG-2 and Gd-PPG-3 were subjected to a parallel test for three times). As shown in FIG. 26A and FIG. 26B, the relationship of 1/Tᵢ changing with the gadolinium concentration is drawn, and a slope of the fitted straight line is the Tᵢ relaxation rate (i=1 or 2). It can be seen that in the 3.0 T MRI scanning system, the r₁ value of the gadolinium chelate Gd-PPG is 35.94±0.76 mM⁻¹ s⁻¹, the r₂ value is 57.52±0.90 mM⁻¹ s⁻¹, and r₂/r₁ is 1.60±0.01. The r₁ value is much higher than the r₁ value (r₁=4-7 mM⁻¹ s⁻¹) of the commercially available gadolinium-based contrast agent, and significantly higher than that of the highly toxic free gadolinium ion solution. In addition, the gadolinium chelate Gd-PPG also has a lower r₂/r₁ ratio, showing a better MRI contrast effect.

### Example 21

Preparation of Gadolinium Chelate (Gd-PTMG)

40 mL of polytetramethylene glycol (M_{w}=2000) solution with a concentration of 4.0 mg/mL was prepared, and the polymer solution was heated at 100° C. for refluxing. 0.8 mL of gadolinium nitrate solution with a concentration of 125 mM was immediately added to the reaction system, and the reaction continued for 60 minutes at 100° C. under magnetic stirring. After being cooled to the room temperature, the solution was purified by means of membrane dialysis to obtain a product labeled as Gd-PTMG finally.

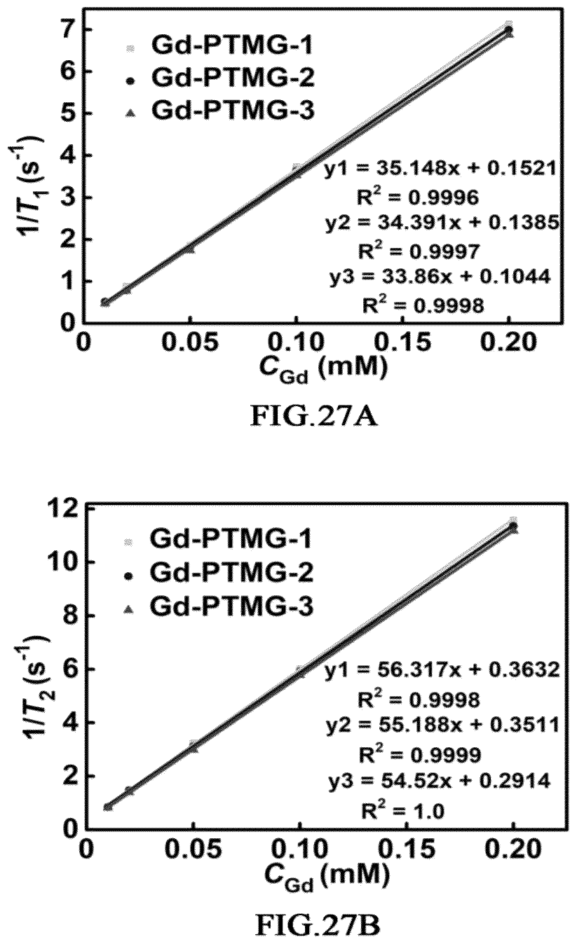

Characterization and performance tests were performed on a sample provided in Example 21. The gadolinium recovery rate of the sample prepared in Example 21 was 86.4%, indicating that the utilization rate of raw materials was higher. The sample prepared in Example 21 was prepared into at least 5 aqueous solutions with different concentrations, in vitro imaging was performed by means of the 3.0 T clinical MRI system (Philips, Ingenia), and a longitudinal relaxation time and a transverse relaxation time (T₁, T₂) thereof were measured (three samples Gd-PTMG-1, Gd-PTMG-2 and Gd-PTMG-3 were subjected to a parallel test for three times). As shown in FIG. 27A and FIG. 27B, the relationship of 1/Tᵢ changing with the gadolinium concentration is drawn, and a slope of the fitted straight line is the Tᵢ relaxation rate (i=1 or 2). It can be seen that in the 3.0 T MRI scanning system, the r₁ value of the gadolinium chelate Gd-PTMG is 34.47±0.65 mM⁻¹ s⁻¹, the r₂ value is 55.34±0.92 mM⁻¹ s⁻¹, and r₂/r₁ is 1.61±0.00. The r₁ value is much higher than the r₁ value (r₁=4-7 mM⁻¹ s⁻¹) of the commercially available gadolinium-based contrast agent, and significantly higher than that of the highly toxic free gadolinium ion solution. In addition, the gadolinium chelate Gd-PTMG also has a lower r₂/r₁ ratio, showing a better MRI contrast effect.

### Example 22

Preparation of Gadolinium Chelate (Gd-PolyQ)

40 mL of polyglutamine (M_{w}=2000) solution with a concentration of 4.0 mg/mL was prepared, and the polymer solution was heated at 100° C. for refluxing. 0.8 mL of gadolinium nitrate solution with a concentration of 125 mM was immediately added to the reaction system, and the reaction continued for 60 minutes at 100° C. under the magnetic stirring. After being cooled to the room temperature, the solution was purified by means of membrane dialysis to obtain a product labeled as Gd-PolyQ finally.

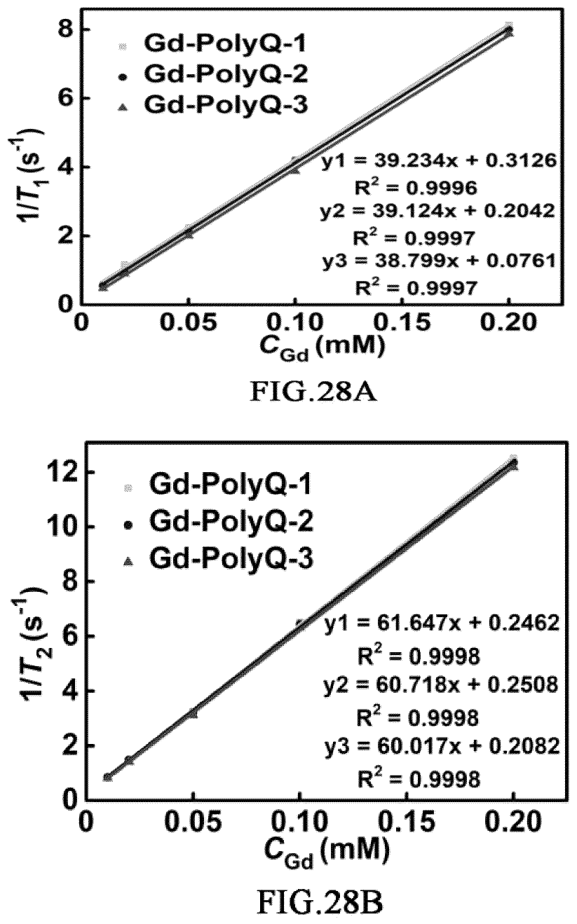

Characterization and performance tests were performed on a sample provided in Example 22. The gadolinium recovery rate of the sample prepared in Example 22 was 88.7%, indicating that the utilization rate of raw materials was higher. The sample prepared in Example 22 was prepared into at least 5 aqueous solutions with different concentrations, in vitro imaging was performed by means of the 3.0 T clinical MRI system (Philips, Ingenia), and a longitudinal relaxation time and a transverse relaxation time (T₁, T₂) thereof were measured (three samples Gd-PolyQ-1, Gd-PolyQ-2 and Gd-PolyQ-3 were subjected to a parallel test for three times). As shown in FIG. 28A and FIG. 28B, the relationship of 1/Tᵢ changing with the gadolinium concentration is drawn, and a slope of the fitted straight line is the Tᵢ relaxation rate (i=1 or 2). It can be seen that in the 3.0 T MRI scanning system, the r₁ value of the gadolinium chelate Gd-PolyQ is 39.05±0.23 mM⁻¹ s⁻¹, the r₂ value is 60.79±0.82 mM⁻¹ s⁻¹, and r₂/r₁ is 1.56±0.01. The r₁ value is much higher than the r₁ value (r₁=4-7 mM⁻¹ s⁻¹) of the commercially available gadolinium-based contrast agent, and significantly higher than that of the highly toxic free gadolinium ion solution. In addition, the gadolinium chelate Gd-PolyQ also has a lower r₂/r₁ ratio, showing a better MRI contrast effect.

### Example 23

Preparation of Gadolinium Chelate (Gd-PHEA)

40 mL of polyasparagine (M_{w}=2000) solution with a concentration of 4.0 mg/mL was prepared, and the polymer solution was heated at 100° C. for refluxing. 0.8 mL of gadolinium nitrate solution with a concentration of 125 mM was immediately added to the reaction system, and the reaction continued for 60 minutes at 100° C. under the magnetic stirring. After being cooled to the room temperature, the solution was purified by means of membrane dialysis to obtain a product labeled as Gd-PHEA finally.

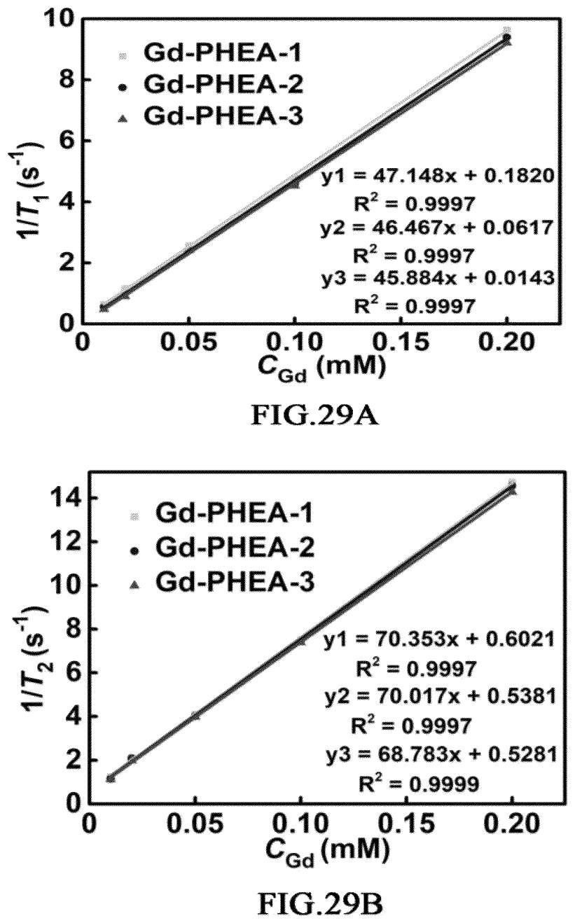

Characterization and performance tests were performed on a sample provided in Example 23. The gadolinium recovery rate of the sample prepared in Example 23 was 91.2%, indicating that the utilization rate of raw materials was higher. The sample prepared in Example 23 was prepared into at least 5 aqueous solutions with different concentrations, in vitro imaging was performed by means of the 3.0 T clinical MRI system (Philips, Ingenia), and a longitudinal relaxation time and a transverse relaxation time (T₁, T₂) thereof were measured (three samples Gd-PHEA-1, Gd-PHEA-2 and Gd-PHEA-3 were subjected to a parallel test for three times). As shown in FIG. 29A and FIG. 29B, the relationship of 1/Tᵢ changing with the gadolinium concentration is drawn, and a slope of the fitted straight line is the Tᵢ relaxation rate (i=1 or 2). It can be seen that in the 3.0 T MRI scanning system, the r₁ value of the gadolinium chelate Gd-PHEA is 46.50±0.63 mM⁻¹ s⁻¹, the r₂ value is 69.72±0.83 mM⁻¹ s⁻¹, and r₂/r₁ is 1.50±0.01. The r₁ value is much higher than the r₁ value (r₁=4-7 mM⁻¹ s⁻¹) of the commercially available gadolinium-based contrast agent, and significantly higher than that of the highly toxic free gadolinium ion solution. In addition, the gadolinium chelate Gd-PHEA also has a lower r₂/r₁ ratio, showing a better MRI contrast effect.

### Example 24

Preparation of Gadolinium Chelate (Gd-PAM)

40 mL of polyacrylamide (M_{w}=2000) solution with a concentration of 4.0 mg/mL was prepared, and the polymer solution was heated at 100° C. for refluxing. 0.8 mL of gadolinium nitrate solution with a concentration of 125 mM was immediately added to the reaction system, and the reaction continued for 60 minutes at 100° C. under the magnetic stirring. After being cooled to the room temperature, the solution was purified by means of membrane dialysis to obtain a product labeled as Gd-PAM finally.

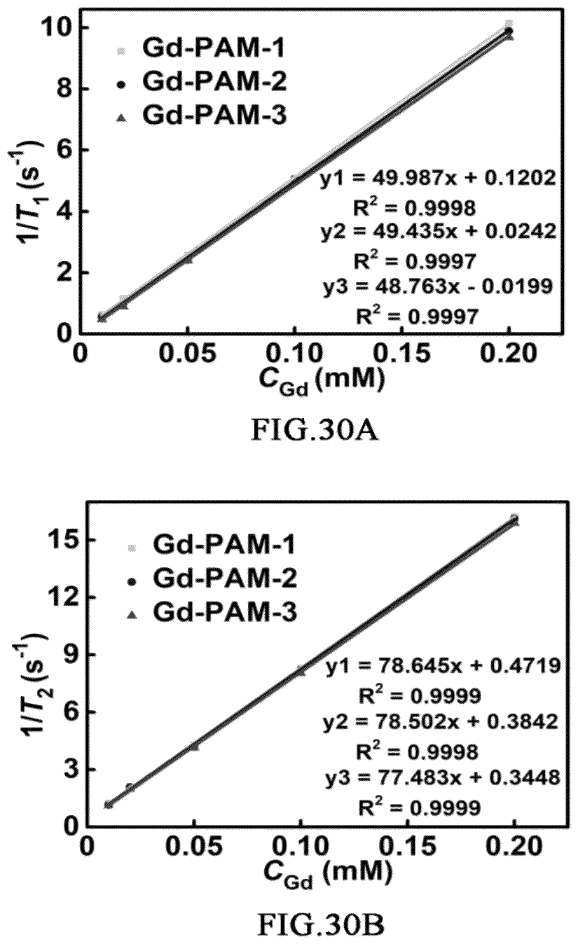

Characterization and performance tests were performed on a sample provided in Example 24. The gadolinium recovery rate of the sample prepared in Example 24 was 90.6%, indicating that the utilization rate of raw materials was higher. The sample prepared in Example 24 was prepared into at least 5 aqueous solutions with different concentrations, in vitro imaging was performed by means of the 3.0 T clinical MRI system (Philips, Ingenia), and a longitudinal relaxation time and a transverse relaxation time (T₁, T₂) thereof were measured (three samples Gd-PAM-1, Gd-PAM-2 and Gd-PAM-3 were subjected to a parallel test for three times). As shown in FIG. 30A and FIG. 30B, the relationship of 1/Tᵢ changing with the gadolinium concentration is drawn, and a slope of the fitted straight line is the Tᵢ relaxation rate (i=1 or 2). It can be seen that in the 3.0 T MRI scanning system, the r₁ value of the gadolinium chelate Gd-PAM is 49.40±0.61 mM⁻¹ s⁻¹, the r₂ value is 78.21±0.63 mM⁻¹ s⁻¹, and r₂/r₁ is 1.58±0.01. The r₁ value is much higher than the r₁ value (r₁=4-7 mM⁻¹ s⁻¹) of the commercially available gadolinium-based contrast agent, and significantly higher than that of the highly toxic free gadolinium ion solution. In addition, the gadolinium chelate Gd-PAM also has a lower r₂/r₁ ratio, showing a better MRI contrast effect.

### Example 25

Preparation of Gadolinium Chelate (Gd-PMAM)

40 mL of polymethacrylamide (M_{w}=2000) solution with a concentration of 4.0 mg/mL was prepared, and the polymer solution was heated at 100° C. for refluxing. 0.8 mL of gadolinium nitrate solution with a concentration of 125 mM was immediately added to the reaction system, and the reaction continued for 60 minutes at 100° C. under the magnetic stirring. After being cooled to the room temperature, the solution was purified by means of membrane dialysis to obtain a product labeled as Gd-PMAM finally.

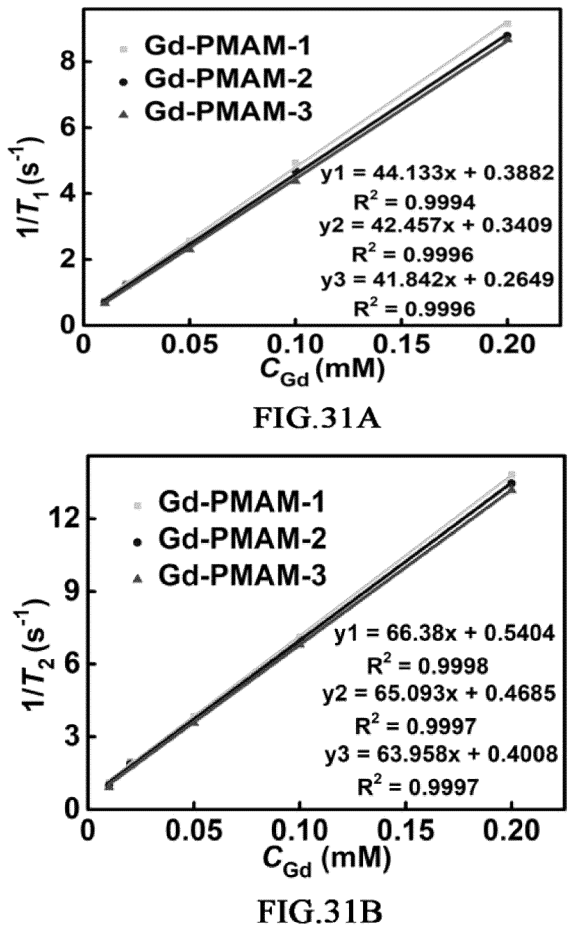

Characterization and performance tests were performed on a sample provided in Example 25. The gadolinium recovery rate of the sample prepared in Example 25 was 87.7%, indicating that the utilization rate of raw materials was higher. The sample prepared in Example 25 was prepared into at least 5 aqueous solutions with different concentrations, in vitro imaging was performed by means of the 3.0 T clinical MRI system (Philips, Ingenia), and a longitudinal relaxation time and a transverse relaxation time (T₁, T₂) thereof were measured (three samples Gd-PMAM-1, Gd-PMAM-2 and Gd-PMAM-3 were subjected to a parallel test for three times). As shown in FIG. 31A and FIG. 31B, the relationship of 1/Tᵢ changing with the gadolinium concentration is drawn, and a slope of the fitted straight line is the Tᵢ relaxation rate (i=1 or 2). It can be seen that in the 3.0 T MRI scanning system, the r₁ value of the gadolinium chelate Gd-PMAM is 42.81±1.19 mM⁻¹ s⁻¹, the r₂ value is 65.14±1.21 mM⁻¹ s⁻¹, and r₂/r₁ is 1.52±0.02. The r₁ value is much higher than the r₁ value (r₁=4-7 mM⁻¹ s⁻¹) of the commercially available gadolinium-based contrast agent, and significantly higher than that of the highly toxic free gadolinium ion solution. In addition, the gadolinium chelate Gd-PMAM also has a lower r₂/r₁ ratio, showing a better MRI contrast effect.

### Example 26

Preparation of Gadolinium Chelate (Gd-HSA)

40 mL of human serum albumin (M_{w}=2000) solution with a concentration of 4.0 mg/mL was prepared, and the polymer solution was heated at 100° C. for refluxing. 0.8 mL of gadolinium nitrate solution with a concentration of 125 mM was immediately added to the reaction system, and the reaction continued for 60 minutes at 100° C. under the magnetic stirring. After being cooled to the room temperature, the solution was purified by means of membrane dialysis to obtain a product labeled as Gd-HSA finally.

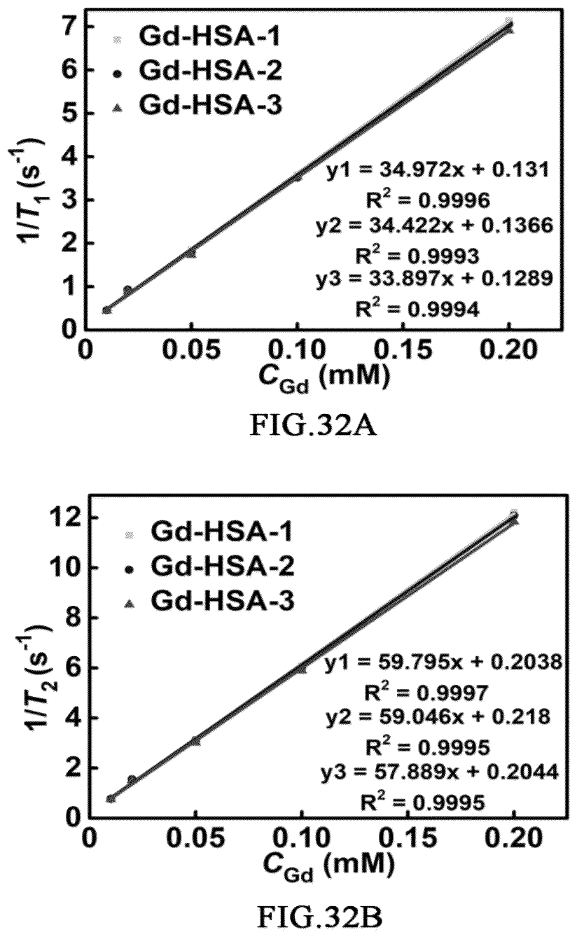

Characterization and performance tests were performed on a sample provided in Example 26. The gadolinium recovery rate of the sample prepared in Example 26 was 90.2%, indicating that the utilization rate of raw materials was higher. The sample prepared in Example 26 was prepared into at least 5 aqueous solutions with different concentrations, in vitro imaging was performed by means of the 3.0 T clinical MRI system (Philips, Ingenia), and a longitudinal relaxation time and a transverse relaxation time (T₁, T₂) thereof were measured (three samples Gd-HSA-1, Gd-HSA-2 and Gd-HSA-3 were subjected to a parallel test for three times). As shown in FIG. 32A and FIG. 32B, the relationship of 1/Tᵢ changing with the gadolinium concentration is drawn, and a slope of the fitted straight line is the Tᵢ relaxation rate (i=1 or 2). It can be seen that in the 3.0 T MRI scanning system, the r₁ value of the gadolinium chelate Gd-HSA is 34.43±0.54 mM⁻¹ s⁻¹, the r₂ value is 59.92±1.57 mM⁻¹ s⁻¹, and r₂/r₁ is 1.74±0.02. The r₁ value is much higher than the r₁ value (r₁=4-7 mM⁻¹ s⁻¹) of the commercially available gadolinium-based contrast agent, and significantly higher than that of the highly toxic free gadolinium ion solution. In addition, the gadolinium chelate Gd-HSA also has a lower r₂/r₁ ratio, showing a better MRI contrast effect.

### Example 27

Preparation of Gadolinium Chelate (Gd-BSA)

40 mL of bovine serum albumin (M_{w}=2000) solution with a concentration of 4.0 mg/mL was prepared, and the polymer solution was heated at 100° C. for refluxing. 0.8 mL of gadolinium nitrate solution with a concentration of 125 mM was immediately added to the reaction system, and the reaction continued for 60 minutes at 100° C. under the magnetic stirring. After being cooled to the room temperature, the solution was purified by means of membrane dialysis to obtain a product labeled as Gd-BSA finally.

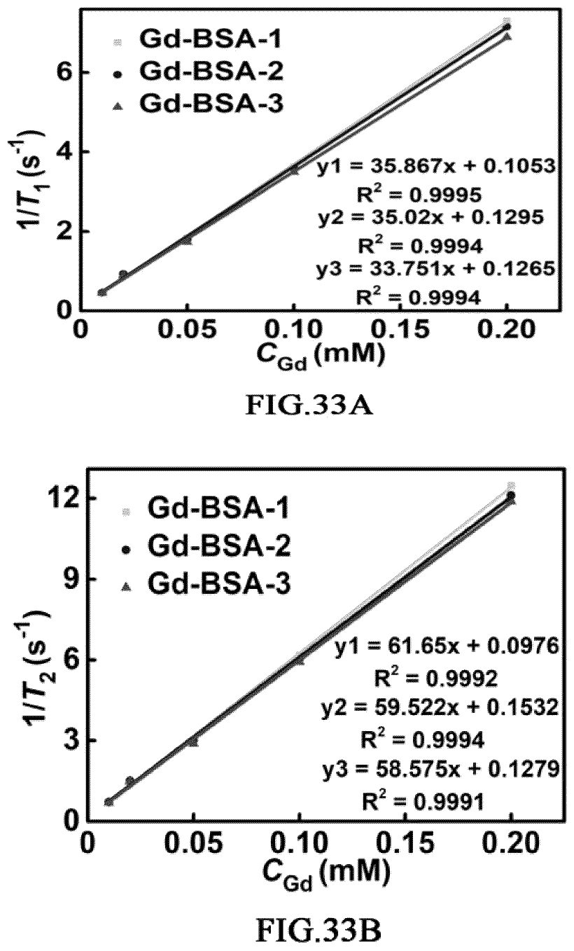

Characterization and performance tests were performed on a sample provided in Example 27. The gadolinium recovery rate of the sample prepared in Example 27 was 85.6%, indicating that the utilization rate of raw materials was higher. The sample prepared in Example 27 was prepared into at least 5 aqueous solutions with different concentrations, in vitro imaging was performed by means of the 3.0 T clinical MRI system (Philips, Ingenia), and a longitudinal relaxation time and a transverse relaxation time (T₁, T₂) thereof were measured (three samples Gd-BSA-1, Gd-BSA-2 and Gd-BSA-3 were subjected to a parallel test for three times). As shown in FIG. 33A and FIG. 33B, the relationship of 1/Tᵢ changing with the gadolinium concentration is drawn, and a slope of the fitted straight line is the Tᵢ relaxation rate (i=1 or 2). It can be seen that in the 3.0 T MRI scanning system, the r₁ value of the gadolinium chelate Gd-BSA is 34.88±1.06 mM⁻¹ s⁻¹, the r₂ value is 59.92±1.57 mM⁻¹ s⁻¹, and r₂/r₁ is 1.72±0.02. The r₁ value is much higher than the r₁ value (r₁=4-7 mM⁻¹ s⁻¹) of the commercially available gadolinium-based contrast agent, and significantly higher than that of the highly toxic free gadolinium ion solution. In addition, the gadolinium chelate Gd-BSA also has a lower r₂/r₁ ratio, showing a better MRI contrast effect.

### Example 28

Preparation of Gadolinium Chelate (Gd-RGD)

40 mL of a RGD peptide (M_{w}=2000) solution with a concentration of 4.0 mg/mL was prepared, and the polymer solution was heated at 100° C. for refluxing. 0.8 mL of gadolinium nitrate solution with a concentration of 125 mM was immediately added to the reaction system, and the reaction continued for 60 minutes at 100° C. under the magnetic stirring. After being cooled to the room temperature, the solution was purified by means of membrane dialysis to obtain a product labeled as Gd-RGD finally.

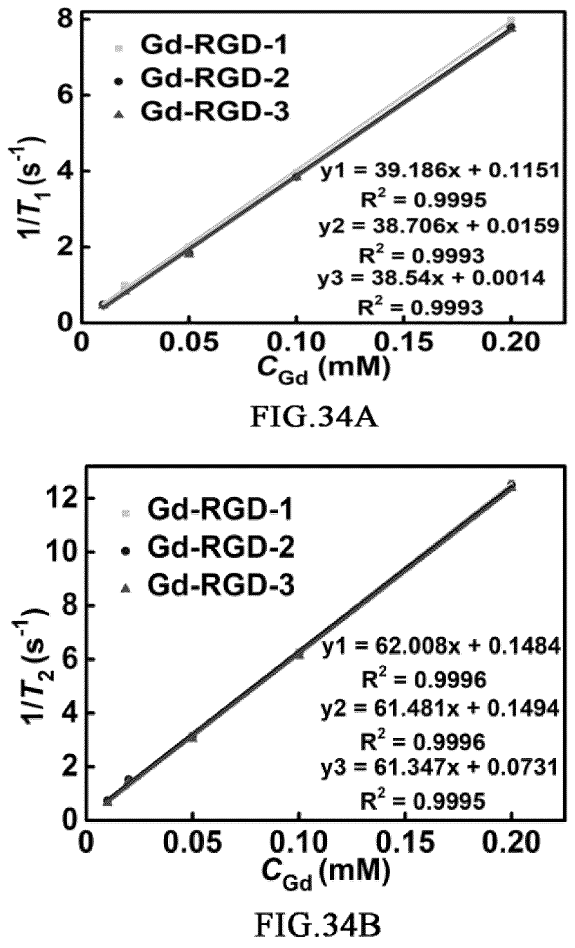

Characterization and performance tests were performed on a sample provided in Example 28. The gadolinium recovery rate of the sample prepared in Example 28 was 82.0%, indicating that the utilization rate of raw materials was higher. The sample prepared in Example 28 was prepared into at least 5 aqueous solutions with different concentrations, in vitro imaging was performed by means of the 3.0 T clinical MRI system (Philips, Ingenia), and a longitudinal relaxation time and a transverse relaxation time (T₁, T₂) thereof were measured (three samples Gd-RGD-1, Gd-RGD-2 and Gd-RGD-3 were subjected to a parallel test for three times). As shown in FIG. 34A and FIG. 34B, the relationship of 1/Tᵢ changing with the gadolinium concentration is drawn, and a slope of the fitted straight line is the Tᵢ relaxation rate (i=1 or 2). It can be seen that in the 3.0 T MRI scanning system, the r₁ value of the gadolinium chelate Gd-RGD is 38.81±0.34 mM⁻¹ s⁻¹, the r₂ value is 61.61±0.35 mM⁻¹ s⁻¹, and r₂/r₁ is 1.59±0.01. The r₁ value is much higher than the r₁ value (r₁=4-7 mM⁻¹ s⁻¹) of the commercially available gadolinium-based contrast agent, and significantly higher than that of the highly toxic free gadolinium ion solution. In addition, the gadolinium chelate Gd-RGD also has a lower r₂/r₁ ratio, showing a better MRI contrast effect.

### Example 29

Preparation of Gadolinium Chelate (Gd-AB)

40 mL of a 0-amyloid polypeptide (M_{w}=2000) solution with a concentration of 4.0 mg/mL was prepared, and the polymer solution was heated at 100° C. for refluxing. 0.8 mL of gadolinium nitrate solution with a concentration of 125 mM was immediately added to the reaction system, and the reaction continued for 60 minutes at 100° C. under the magnetic stirring. After being cooled to the room temperature, the solution was purified by means of membrane dialysis to obtain a product labeled as Gd-Aβ finally.

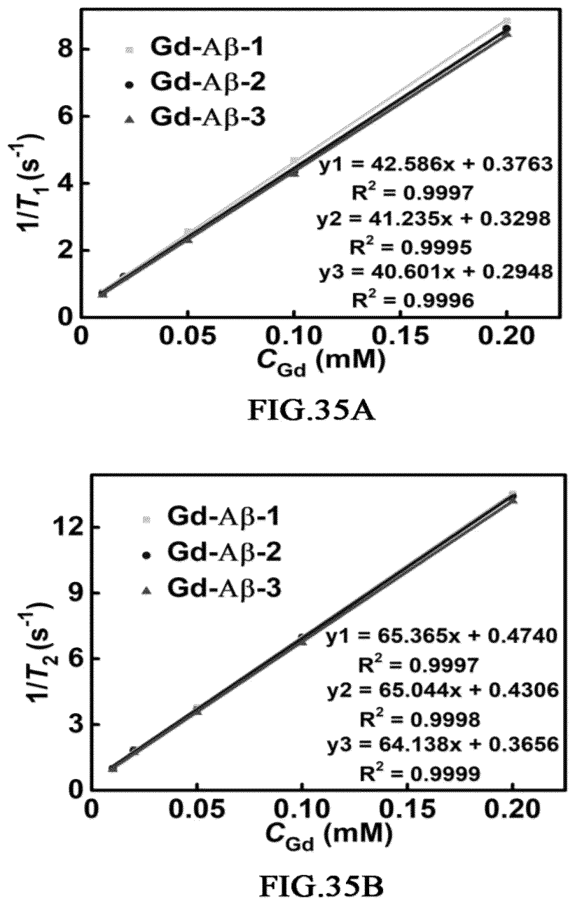

Characterization and performance tests were performed on a sample provided in Example 29. The gadolinium recovery rate of the sample prepared in Example 29 was 84.4%, indicating that the utilization rate of raw materials was higher. The sample prepared in Example 29 was prepared into at least 5 aqueous solutions with different concentrations, in vitro imaging was performed by means of the 3.0 T clinical MRI system (Philips, Ingenia), and a longitudinal relaxation time and a transverse relaxation time (T₁, T₂) thereof were measured (three samples Gd-Aβ-1, Gd-Aβ-2 and Gd-Aβ-3 were subjected to a parallel test for three times). As shown in FIG. 35A and FIG. 35B, the relationship of 1/Tᵢ changing with the gadolinium concentration is drawn, and a slope of the fitted straight line is the Tᵢ relaxation rate (i=1 or 2). It can be seen that in the 3.0 T MRI scanning system, the r₁ value of the gadolinium chelate Gd-Aβ is 41.47±1.01 mM⁻¹ s⁻¹, the r₂ value is 64.85±0.64 mM⁻¹ s⁻¹, and r₂/r₁ is 1.56±0.03. The r₁ value is much higher than the r₁ value (r₁=4-7 mM⁻¹ s⁻¹) of the commercially available gadolinium-based contrast agent, and significantly higher than that of the highly toxic free gadolinium ion solution. In addition, the gadolinium chelate Gd-Aβ also has a lower r₂/r₁ ratio, showing a better MRI contrast effect.

### Example 30

Preparation of Gadolinium Chelate (Gd-CS)

40 mL of chitosan (M_{w}=2000) solution with a concentration of 4.0 mg/mL was prepared, and the polymer solution was heated at 100° C. for refluxing. 0.8 mL of gadolinium nitrate solution with a concentration of 125 mM was immediately added to the reaction system, and the reaction continued for 60 minutes at 100° C. under the magnetic stirring. After being cooled to the room temperature, the solution was purified by means of membrane dialysis to obtain a product labeled as Gd-CS finally.

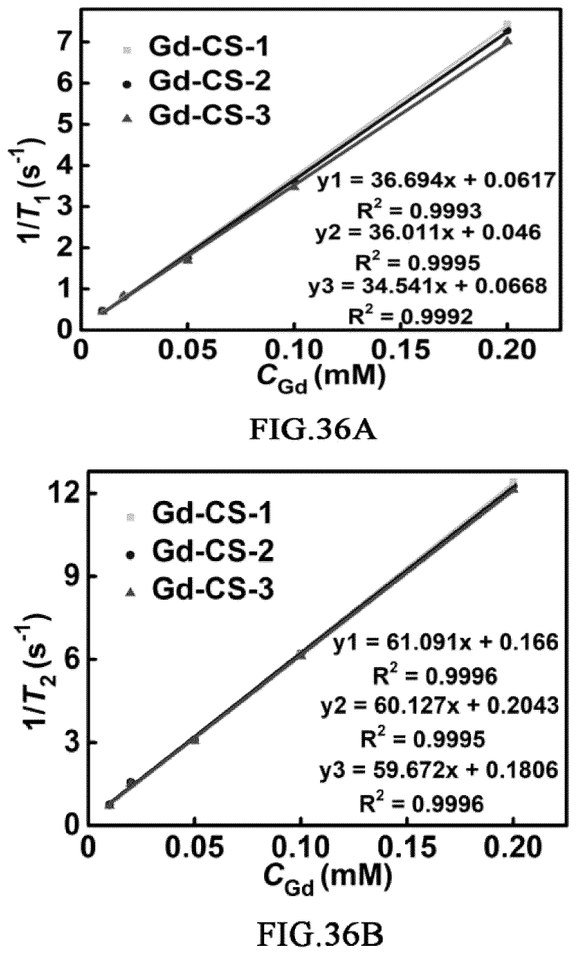

Characterization and performance tests were performed on a sample provided in Example 30. The gadolinium recovery rate of the sample prepared in Example 30 was 83.6%, indicating that the utilization rate of raw materials was higher. The sample prepared in Example 30 was prepared into at least 5 aqueous solutions with different concentrations, in vitro imaging was performed by means of the 3.0 T clinical MRI system (Philips, Ingenia), and a longitudinal relaxation time and a transverse relaxation time (T₁, T₂) thereof were measured (three samples Gd-CS-1, Gd-CS-2 and Gd-CS-3 were subjected to a parallel test for three times). As shown in FIG. 36A and FIG. 36B, the relationship of 1/Tᵢ changing with the gadolinium concentration is drawn, and a slope of the fitted straight line is the Tᵢ relaxation rate (i=1 or 2). It can be seen that in the 3.0 T MRI scanning system, the r₁ value of the gadolinium chelate Gd-CS is 35.75±1.10 mM⁻¹ s⁻¹, the r₂ value is 60.30±0.72 mM⁻¹ s⁻¹, and r₂/r₁ is 1.69±0.03. The r₁ value is much higher than the r₁ value (r₁=4-7 mM⁻¹ s⁻¹) of the commercially available gadolinium-based contrast agent, and significantly higher than that of the highly toxic free gadolinium ion solution. In addition, the gadolinium chelate Gd-CS also has a lower r₂/r₁ ratio, showing a better MRI contrast effect.

### Example 31

Preparation of Gadolinium Chelate (Gd-SA)

40 mL of sodium alginate (M_{w}=2000) solution with a concentration of 4.0 mg/mL was prepared, and the polymer solution was heated at 100° C. for refluxing. 0.8 mL of gadolinium nitrate solution with a concentration of 125 mM was immediately added to the reaction system, and the reaction continued for 60 minutes at 100° C. under the magnetic stirring. After being cooled to the room temperature, the solution was purified by means of membrane dialysis to obtain a product labeled as Gd-SA finally.

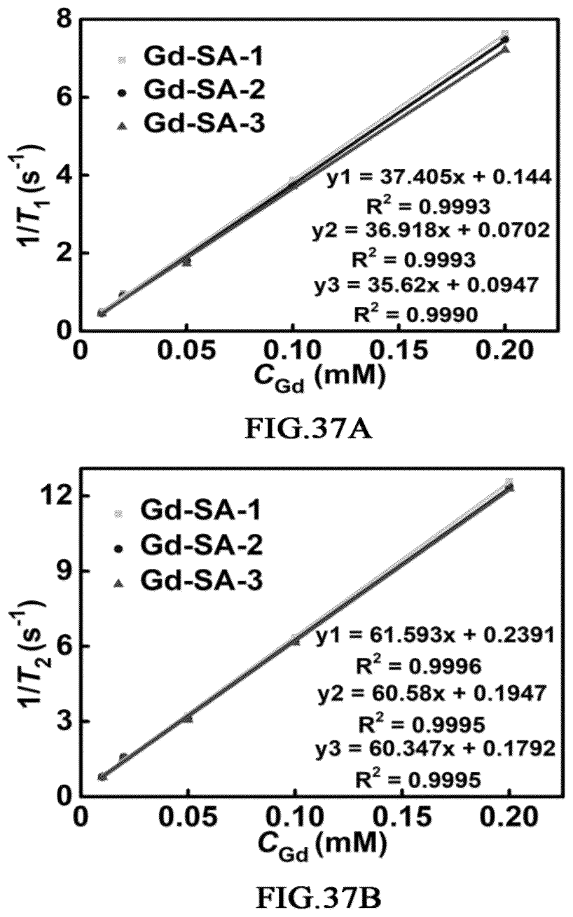

Characterization and performance tests were performed on a sample provided in Example 31. The gadolinium recovery rate of the sample prepared in Example 31 was 82.5%, indicating that the utilization rate of raw materials was higher. The sample prepared in Example 31 was prepared into at least 5 aqueous solutions with different concentrations, in vitro imaging was performed by means of the 3.0 T clinical MRI system (Philips, Ingenia), and a longitudinal relaxation time and a transverse relaxation time (T₁, T₂) thereof were measured (three samples Gd-SA-1, Gd-SA-2 and Gd-SA-3 were subjected to a parallel test for three times). As shown in FIG. 37A and FIG. 37B, the relationship of 1/Tᵢ changing with the gadolinium concentration is drawn, and a slope of the fitted straight line is the Tᵢ relaxation rate (i=1 or 2). It can be seen that in the 3.0 T MRI scanning system, the r₁ value of the gadolinium chelate Gd-SA is 36.65±0.92 mM⁻¹ s⁻¹, the r₂ value is 60.84±0.66 mM⁻¹ s⁻¹, and r₂/r₁ is 1.67±0.03. The r₁ value is much higher than the r₁ value (r₁=4-7 mM⁻¹ s⁻¹) of the commercially available gadolinium-based contrast agent, and significantly higher than that of the highly toxic free gadolinium ion solution. In addition, the gadolinium chelate Gd-SA also has a lower r₂/r₁ ratio, showing a better MRI contrast effect.

### Example 32

Preparation of Gadolinium Chelate (Gd-PAA-PLA)

40 mL of polyacrylic acid-polylactic acid (M_{w}=2000) copolymer solution with a concentration of 4.0 mg/mL was prepared, and the polymer solution was heated at 100° C. for refluxing. 0.8 mL of gadolinium nitrate solution with a concentration of 125 mM was immediately added to the reaction system, and the reaction continued for 60 minutes at 100° C. under the magnetic stirring. After being cooled to the room temperature, the solution was purified by means of membrane dialysis to obtain a product labeled as Gd-PAA-PLA finally.

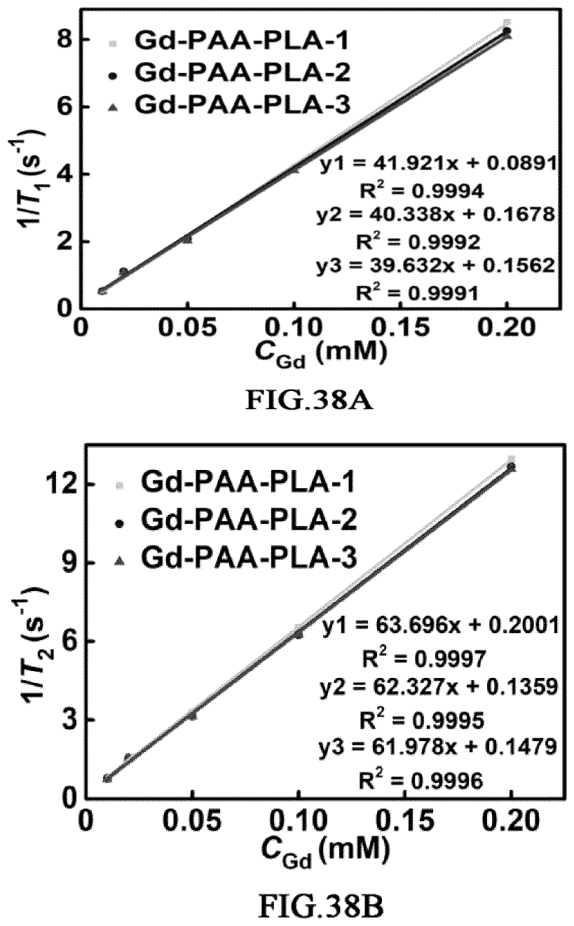

Characterization and performance tests were performed on a sample provided in Example 32. The gadolinium recovery rate of the sample prepared in Example 32 was 88.6%, indicating that the utilization rate of raw materials was higher. The sample prepared in Example 32 was prepared into at least 5 aqueous solutions of different concentrations, in vitro imaging was performed by means of the 3.0 T clinical MRI system, and a longitudinal relaxation time and transverse relaxation time (T₁, T₂) were measured (three samples Gd-PAA-PLA-1, Gd-PAA-PLA-2 and Gd-PAA-PLA-3 were subjected to a parallel test for three times). As shown in FIG. 38A and FIG. 38B, the relationship of 1/Tᵢ changing with the gadolinium concentration is drawn, and a slope of the fitted straight line is the Tᵢ relaxation rate (i=1 or 2). It can be seen that in the 3.0 T MRI scanning system (Philips, Ingenia), the r₁ value of the gadolinium chelate Gd-PAA-PLA is 40.63±1.17 mM⁻¹ s⁻¹, the r₂ value is 62.67±0.91 mM⁻¹ s⁻¹, and r₂/r₁ is 1.54±0.02. The r₁ value is much higher than the r₁ value (r₁=4-7 mM⁻¹ s⁻¹) of the commercially available gadolinium-based contrast agent, and significantly higher than that of the highly toxic free gadolinium ion solution. In addition, the gadolinium chelate Gd-PAA-PLA also has a lower r₂/r₁ ratio, showing a better MRI contrast effect.

### Example 33

Preparation of Gadolinium Chelate (Gd-PGA-PEG)

40 mL of polyglycolic acid-polyethylene glycol (M_{w}=2000) copolymer solution with a concentration of 4.0 mg/mL was prepared, and the polymer solution was heated at 100° C. for refluxing. 0.8 mL of gadolinium nitrate solution with a concentration of 125 mM was immediately added to the reaction system, and the reaction continued for 60 minutes at 100° C. under the magnetic stirring. After being cooled to the room temperature, the solution was purified by means of membrane dialysis to obtain a product labeled as Gd-PGA-PEG finally.

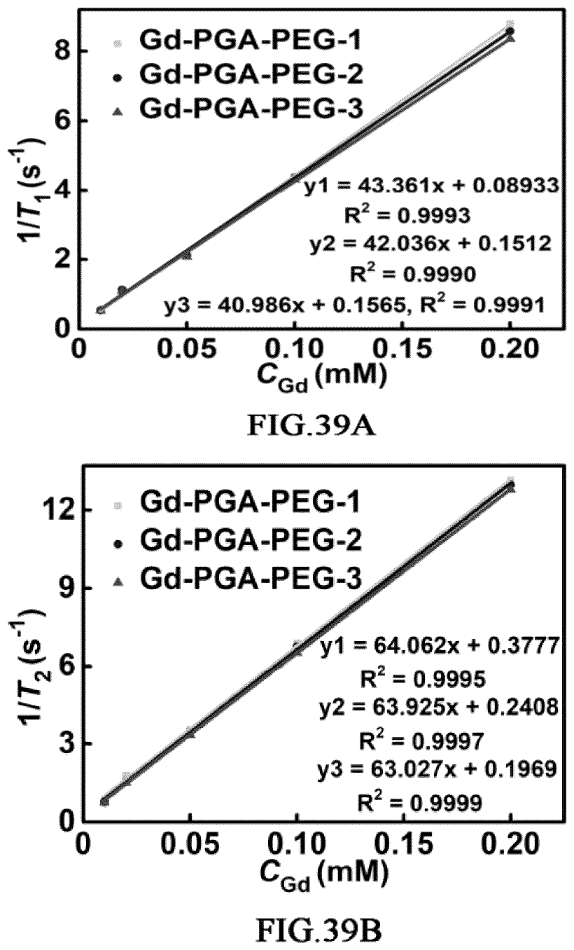

Characterization and performance tests were performed on a sample provided in Example 33. The gadolinium recovery rate of the sample prepared in Example 33 was 92.3%, indicating that the utilization rate of raw materials was higher. The sample prepared in Example 33 was prepared into at least 5 aqueous solutions with different concentrations, in vitro imaging was performed by means of the 3.0 T clinical MRI system (Philips, Ingenia), and a longitudinal relaxation time and a transverse relaxation time (T₁, T₂) thereof were measured (three samples Gd-PGA-PEG-1, Gd-PGA-PEG-2 and Gd-PGA-PEG-3 were subjected to a parallel test for three times). As shown in FIG. 39A and FIG. 39B, the relationship of 1/Tᵢ changing with the gadolinium concentration is drawn, and a slope of the fitted straight line is the Tᵢ relaxation rate (i=1 or 2). It can be seen that in the 3.0 T MRI scanning system (Philips, Ingenia), the r₁ value of the gadolinium chelate Gd-PGA-PEG is 42.13±1.19 mM⁻¹ s⁻¹, the r₂ value is 63.67±0.56 mM⁻¹ s⁻¹, and r₂/r₁ is 1.51±0.03. The r₁ value is much higher than the r₁ value (r₁=4-7 mM⁻¹ s⁻¹) of the commercially available gadolinium-based contrast agent, and significantly higher than that of the highly toxic free gadolinium ion solution. In addition, the gadolinium chelate Gd-PGA-PEG also has a lower r₂/r₁ ratio, showing a better MRI contrast effect.

### Example 34

Preparation of Gadolinium Chelate (Gd-PAA/PASP)

40 mL of polyacrylic acid/polyaspartic acid (1:1) (the M_{W} of both are 2000) mixture solution with a concentration of 4.0 mg/mL was prepared, and the polymer solution was heated at 100° C. for refluxing. 0.8 mL of gadolinium nitrate solution with a concentration of 125 mM was immediately added to the reaction system, and the reaction continued for 60 minutes at 100° C. under the magnetic stirring. After being cooled to the room temperature, the solution was purified by means of membrane dialysis to obtain a product labeled as Gd-PAA/PASP finally.

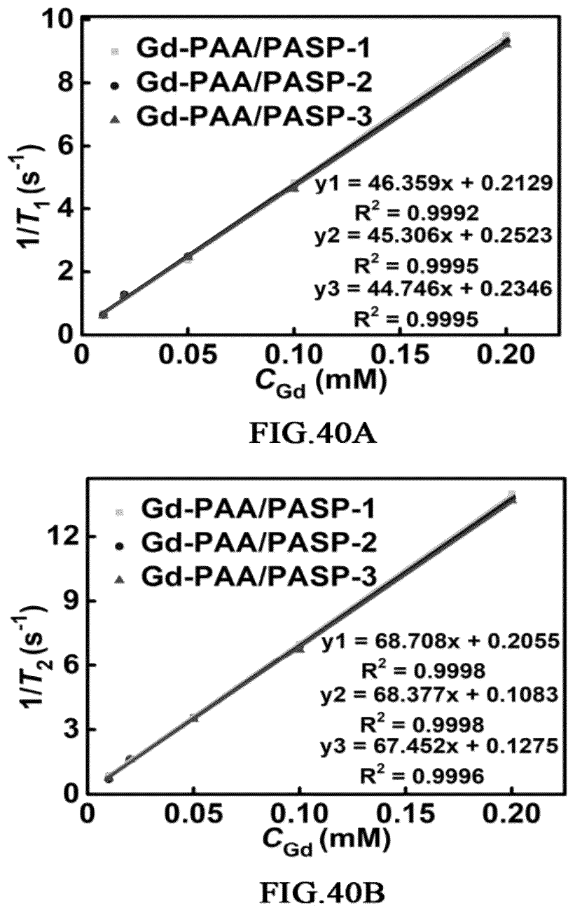

Characterization and performance tests were performed on a sample provided in Example 34. The gadolinium recovery rate of the sample prepared in Example 34 was 92.9%, indicating that the utilization rate of raw materials was higher. The sample prepared in Example 34 was prepared into at least 5 aqueous solutions with different concentrations, in vitro imaging was performed by means of the 3.0 T clinical MRI system (Philips, Ingenia), and a longitudinal relaxation time and a transverse relaxation time (T₁, T₂) thereof were measured (three samples Gd-PAA/PASP-1, Gd-PAA/PASP-2 and Gd-PAA/PASP-3 were subjected to a parallel test for three times). As shown in FIG. 40A and FIG. 40B, the relationship of 1/Tᵢ changing with the gadolinium concentration is drawn, and a slope of the fitted straight line is the Tᵢ relaxation rate (i=1 or 2). It can be seen that in the 3.0 T MRI scanning system, the r₁ value of the gadolinium chelate Gd-PAA/PASP is 45.47±0.82 mM⁻¹ s⁻¹, the r₂ value is 68.18±0.65 mM⁻¹ s⁻¹, and r₂/r₁ is 1.50±0.02. The r₁ value is much higher than the r₁ value (r₁=4-7 mM⁻¹ s⁻¹) of the commercially available gadolinium-based contrast agent, and significantly higher than that of the highly toxic free gadolinium ion solution. In addition, the gadolinium chelate Gd-PAA/PASP also has a lower r₂/r₁ ratio, showing a better MRI contrast effect.

### Example 35

Preparation of Gadolinium Chelate (Gd-γ-PGA/PASP)

40 mL of polyglutamic acid/polyaspartic acid (1:1) (the M_{w} of both are 2000) mixture solution with a concentration of 4.0 mg/mL was prepared, and the polymer solution was heated at 100° C. for refluxing. 0.8 mL of gadolinium nitrate solution with a concentration of 125 mM was immediately added to the reaction system, and the reaction continued for 60 minutes at 100° C. under the magnetic stirring. After being cooled to the room temperature, the solution was purified by means of membrane dialysis to obtain a product labeled as Gd-γ-PGA/PASP finally.

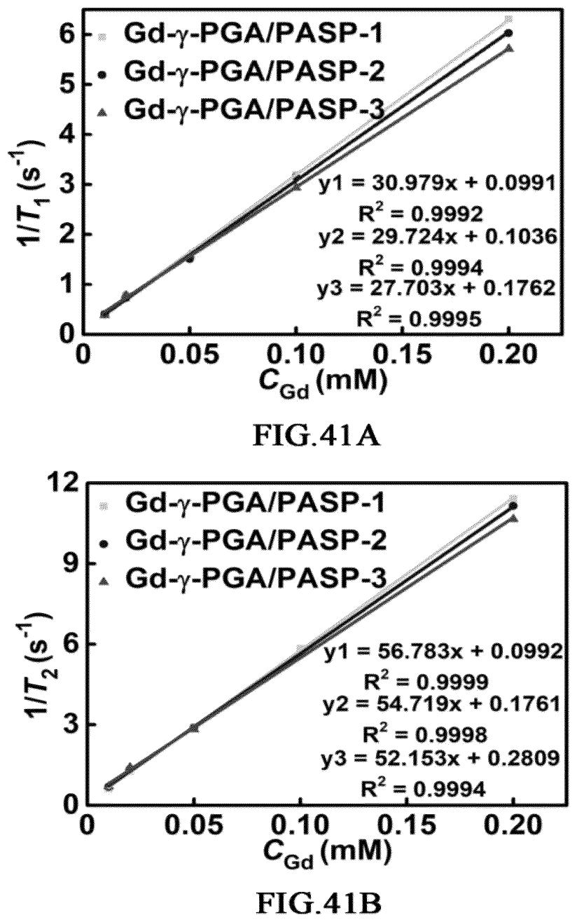

Characterization and performance tests were performed on a sample provided in Example 35. The gadolinium recovery rate of the sample prepared in Example 35 was 82.8%, indicating that the utilization rate of raw materials was higher. The sample prepared in Example 35 was prepared into at least 5 aqueous solutions with different concentrations, in vitro imaging was performed by means of the 3.0 T clinical MRI system (Philips, Ingenia), and a longitudinal relaxation time and a transverse relaxation time (T₁, T₂) thereof were measured (three samples Gd-γ-PGA/PASP-1, Gd-γ-PGA/PASP-2 and Gd-γ-PGA/PASP-3 were subjected to a parallel test for three times). As shown in FIG. 41A and FIG. 41B, the relationship of 1/Tᵢ changing with the gadolinium concentration is drawn, and a slope of the fitted straight line is the Tᵢ relaxation rate (i=1 or 2). It can be seen that in the 3.0 T MRI scanning system (Philips, Ingenia), the r₁ value of the gadolinium chelate Gd-γ-PGA/PASP is 29.47±1.65 mM⁻¹ s⁻¹, the r₂ value is 54.55±2.32 mM⁻¹ s⁻¹, and r₂/r₁ is 1.85±0.03. The r₁ value is much higher than the r₁ value (r₁=4-7 mM⁻¹ s⁻¹) of the commercially available gadolinium-based contrast agent, and significantly higher than that of the highly toxic free gadolinium ion solution. In addition, the gadolinium chelate Gd-γ-PGA/PASP also has a lower r₂/r₁ ratio, showing a better MRI contrast effect.

The examples of the present disclosure are preferred embodiments, but the embodiments of the present disclosure are not limited to the foregoing specific examples. Any changes, modifications, substitutions, combinations, and simplifications thereof made without departing from the spirit and principles of the present disclosure should be equivalent alternatives and fall within the protection scope of the present disclosure.

1. A gadolinium chelate, which is a complex formed by chelation of gadolinium ions and a macromolecule, wherein the macromolecule comprises any one of or a copolymer or mixture of two or more of a carboxylic acid-containing high-molecular polymer, an amino-containing high-molecular polymer, a hydroxyl-containing high-molecular polymer, a polyester high-molecular polymer, a polyether high-molecular polymer, a polyamide high-molecular polymer, a protein, a polypeptide, or a polysaccharide.

2. The gadolinium chelate according to claim 1, wherein a molecular weight of the macromolecule is 1,000-1,000,000.

3. The gadolinium chelate according to claim 1, wherein the carboxylic acid-containing high-molecular polymer comprises any one or more selected from the group consisting of polyacrylic acid, polymaleic acid, polymethacrylic acid, poly(2-ethylacrylic acid), polyglutamic acid, and polyaspartic acid.

4. The gadolinium chelate according to claim 1, wherein the amino-containing high-molecular polymer comprises any one or more selected from the group consisting of polylysine, polyarginine, polyhistidine, and polyethyleneimine.

5. The gadolinium chelate according to claim 1, wherein the hydroxyl-containing high-molecular polymer comprises any one or more selected from the group consisting of polyserine, polythreonine, polytyrosine, and polyvinyl alcohol.

6. The gadolinium chelate according to claim 1, wherein the polyester high-molecular polymer comprises any one or more selected from the group consisting of polylactic acid, polyglycolic acid, polycaprolactone, and poly(2-hydroxyethyl methacrylate).

7. The gadolinium chelate according to claim 1, wherein the polyether high-molecular polymer comprises any one or more selected from the group consisting of polyethylene glycol, polypropylene glycol, and polytetramethylene glycol.

8. The gadolinium chelate according to claim 1, wherein the polyamide high-molecular polymer comprises any one or more selected from the group consisting of polyglutamine, polyasparagine, polyacrylamide, and polymethacrylamide.

9. The gadolinium chelate according to claim 1, wherein the protein comprises any one or more selected from the group consisting of a human protein, an animal protein, a plant protein, and a recombinant protein.

10. The gadolinium chelate according to claim 1, wherein the polypeptide comprises any one or more selected from the group consisting of an RGD peptide and a β-amyloid polypeptide.

11. The gadolinium chelate according to claim 1, wherein the polysaccharide comprises any one or more of selected from the group consisting chitosan and sodium alginate.

12. A preparation method of the gadolinium chelate according to claim 1, comprising: subjecting gadolinium ions and a macromolecule to a chelation reaction to obtain the gadolinium chelate.

13. (canceled)

14. The preparation method according to claim 12, wherein the gadolinium ions are trivalent gadolinium ions.

15. A preparation method of the gadolinium chelate according to claim 1, comprising: mixing a gadolinium ion solution with a macromolecular solution to perform a chelation reaction, so as to obtain the gadolinium chelate.

16. The preparation method according to claim 15, wherein a concentration of the gadolinium ion solution is 10-1000 mM.

17. The preparation method according to claim 15, wherein a concentration of the macromolecular solution is 0.1-50 mg/mL.

18. The preparation method according to claim 15, wherein a volume ratio of the macromolecular solution to the gadolinium ion solution is 1-1000:1.

19. The preparation method according to claim 15, wherein both the gadolinium ion solution and the macromolecular solution are aqueous solutions.

20. The preparation method according to claim 15, wherein the gadolinium ion solution includes any one or more selected from the group consisting of a gadolinium nitrate solution, a gadolinium fluoride solution, a gadolinium chloride solution, a gadolinium bromide solution and a gadolinium iodide solution.

21. A magnetic resonance imaging contrast agent comprising the gadolinium chelate according to claim 1.
